SEQUENCE LISTING (11 sequences)

SEQ ID NO: 1 - protein, 1246 aa, Solanum lycopersicum
MAENEIEEMLEHLRRIKSGGDLDWLDILRIEELEMVLRVFRTFTKYNDVLLPDSLVELTKRAKLIGEILHRLFGRIPHKCKTNLNLERLESHLLEFFQGNTASLSHNYELNNFDLSKYMDCLENFLNDVLMMFLQKDRFFHSREQLAKHRSIKELKIVQKKIRFLKYIYATEINGYVDYEKQECLENRIQFMTNTVGQYCLAVLDYVTEGKLNEENDNFSKPPYLLSLIVLVELEMKKIFHGEVKASKFTQSKTFKDKKLPKGFSHHLHNLLMYLRNKKLENFPNNIAAQNIDVAIEFLLVFLDADVSNHVINGNWLKKVLLKVGAIAGDILYVIQKLLPRSINKDETSNISLCSIQILEKTKDLKAQVETYYKSLKFTPSQFPTFGGLSFLNSLLRKLNEMSTSKSGLGFLMKPLLGNLEKELSSLTSILEKELSSIFRDVVHHEHNIPKDLQRRTINLSYEAEVAIDSILAQYNAFLHIFCSLPTIVKEIKQINAEVTEMWSADIPLNPHYVAAPLKHLPDRHSNLVTDEEVVGFENKAEELIDYLIRGTNELDVVPIVGMGGQGKTTIARKLYNNDIIVSRFDVRAWCIISQTYNRRELLQDIFSQVTGSDDNGATVDVLADMLRRKLMGKRYLIVLDDMWDCMVWDDLRLSFPDDGIRSRIVVTTRLEEVGKQVKYHTDPYSLPFLTTEESCQLLQKKVFQKEDCPPELQDVSQAVAEKCKGLPLVVVLVAGIIKKRKMEESWWNEVKDALFDYLDSEFEEYSLATMQLSFDNLPHCLKPCLLYMGMFSEDARIPASTLISLWIAEGFVENTESGRLMEEEAEGYLMDLISSNLVMLSKRTYKGRVKYCQVHDVVHHFCLEKSREAKFMLAVKGQYIHFQPSDWKGTRVSFSFSEELSKFASLVSKTQKPFHQHLRSLITTNRAKSINDIFSCQISELRLLKVLDLSSYIVEFLSLATFKPLNQLKYLAVQAFEFYFDPGSHLPHIETFIVMNLPYYDILLPVSFWEMKKLRHAHFGKAEFDKQGLSEGSSKLENLRILKNIVGFDRVDVLSRRCPNLQQLQITYFGNNEEPFCPKLENLTQLQQLQLSFARPRTLSGLQLPSNLNKLVLEGIHIGCVIPFIAGLPSLEYLQLHDVCFPQSEEWCLGDITFHKLKLLKLVKLNISRWDVSEESFPLLETLVIKKCIDLEEIPLSFADIPTLEQIKLIGSWKVSLEDSAVRMKEEIKDTEGCDRLHLVKQHSD

SEQ ID NO: 2 - protein, 387 aa, Solanum lycopersicum
FGGLSFLNSLLRKLNEMSTSKSGLGFLMKPLLGNLEKELSSLTSILEKELSSIFRDVVHHEHNIPKDLQRRTINLSYEAEVAIDSILAQYNAFLHIFCSLPTIVKEIKQINAEVTEMWSADIPLNPHYVAAPLKHLPDRHSNLVTDEEVVGFENKAEELIDYLIRGTNELDVVPIVGMGGQGKTTIARKLYNNDIIVSRFDVRAWCIISQTYNRRELLQDIFSQVTGSDDNGATVDVLADMLRRKLMGKRYLIVLDDMWDCMVWDDLRLSFPDDGIRSRIVVTTRLEEVGKQVKYHTDPYSLPFLTTEESCQLLQKKVFQKEDCPPELQDVSQAVAEKCKGLPLVVVLVAGIIKKRKMEESWWNEVKDALFDYLDSEFEEYSLATMQ

SEQ ID NO: 3 - protein, 1244 aa, Solanum sp.
MAENEIEEMLEHLRRIKSGGDLHWLNIFQLEKLEMVLRVFRTFTKYHDVLLPDSLVELTKMAKLTGEVLHRVLGIIPHKCKLNLERLLSHLLEFFQGNTASLSHNYELNDFDLSKYMDCLENFLNDLLMMFFHEGSLCHSIEQLLIHRTIKELKIVQKKIRFLKYIYATEINGYVDYEKQECLETRIQFMTNTVGQYCLALLDYVAGGKLNKENDNFSKLPYLLSLIVFVELEMKKIFHGEVKASKFTQSKTFKDKKLPKGFSHHLHNLLMYLRNKKLENFPNNIAAQNIDVAIEFLLVFLDADVSNHVINGNWLKEVLLKVVAIAGDILYVIQKLLPRSINKDETSNVSLCSIQILEKTKDLKAQVETYYKSLKFTPSQFPTFGGLSFLDSLLRKLNEMSTSKSGLGFLMKPLLGNLEKELSSLTSILEKELSSIFRDVVHHEHSIPKDLQRRTINLAYEAEVAIDSILAQYNVFLHIFCSLPTILKEIKQINAEVTEMWSADIPLNPHYVAAPLKHLQDRHSNLVTDEEVVGFENKAEELIDYLIRGTNELDVVPIVGMGGQGKTTIARKLYNNDIIVSRFDVRAWCIISQTYNRRELLQDIFSQVTGVNDNGATVDDVADMLRRKLMGKRYLIVLDDMWDCMVWDDLRLSFPDSGNRSRIVVTTRLEEVGKQVKYHTDPYSLPFLTTEESCQLLQKKVFQKDDCPPELQDVSQAVAEKCKGLPLVVVLVAGIIKKRKMEESWWNEVKDALFDYIDSEFEEYSLATMQLSFDNLPHCLKPCLLYMGMFSEDARIPASTLISLWIAEGFVENTESGILMEEAAEGYLMDLISSNLVMLSKRSYKGKVKYCQVHDVVHHFCLEKSREAKFMLAVKGQCIQFQPSDWKGTRVSFSFSEELSKFASLVSKTQKPFHQHLRSLITTNRAKSINDIFSCQISELRLLKVLDLSSYIVEFLSLATFKPLNQLKYLAVQAFEFYFDPGSHLPHIETFIVMNLPYYDILLPVSFWEMKKLRHAHFGKAEFDKQGLSEGSSKLENLRILKNIVGFDRVDVLSRRCPNLQQLQITYFGNNEEPFCPKLENLTQLQQLQLSFARPRTLSGLQLPSNLNKLVLEGIHIGCVIPFIAGLPSLEYLQLHDVCFPQSEEWCLGDITFHKLKLLKLVKLNISRWDVSEESFPLLETLVIKKCIDLEEIPLSFADIPTLEQIKLIGSWKVSLEDSAVRMKEEIKDTEGCDRLHLVKQHSD

SEQ ID NO: 4 - protein, 387 aa, Solanum sp.
FGGLSFLDSLLRKLNEMSTSKSGLGFLMKPLLGNLEKELSSLTSILEKELSSIFRDVVHHEHSIPKDLQRRTINLAYEAEVAIDSILAQYNVFLHIFCSLPTILKEIKQINAEVTEMWSADIPLNPHYVAAPLKHLQDRHSNLVTDEEVVGFENKAEELIDYLIRGTNELDVVPIVGMGGQGKTTIARKLYNNDIIVSRFDVRAWCIISQTYNRRELLQDIFSQVTGVNDNGATVDDVADMLRRKLMGKRYLIVLDDMWDCMVWDDLRLSFPDSGNRSRIVVTTRLEEVGKQVKYHTDPYSLPFLTTEESCQLLQKKVFQKDDCPPELQDVSQAVAEKCKGLPLVVVLVAGIIKKRKMEESWWNEVKDALFDYIDSEFEEYSLATMQ

SEQ ID NO: 5 - protein, 387 aa, Solanum lycocarpum
FGGLSFLDSLLRKLNEMSTSKSGLGFLMKPLLGNLEKELSSLTSILEKELSSIFRDVVHHEHNIPKDLQRRTINLAYEAEVAIDSILAQYNAFLHIFCSLPTIVKEIKQINAEVTEMWSADIPLNPHYVAAPLKHLPDRHSNLVTDEEVVGFENKAEELIDYLIRGTNELDVVPIVGMGGQGKTTIARKLYNNDIIVSRFDVRAWCIISQTYNRRELLQDIFSQVTGCNDNGATVDVVADMLRRKLMGKRYLIVLDDMWDCMVWDDLRLSFPDSGNRSRIVVTTRLEEVGKQVKYHTDPYSLPFLTTEESCQLLQKKVFQKEDCPPELQDVSQAVAEKCKGLPLVVVLVAGIIKKRKMEESWWNEVKDALFDYLDSEFEEYSLATMQ

SEQ ID NO: 6 - protein, 12 aa, Solanum lycopersicum
KDRFFHSREQLA

SEQ ID NO: 7 - protein, 12 aa, Solanum sp.
EGSLCHSIEQLL

SEQ ID NO: 8 - protein, 18 aa, Solanum lycopersicum
LQKDRFFHSREQLAKHRS

SEQ ID NO: 9 - protein, 18 aa, Solanum sp.
FHEGSLCHSIEQLLIHRT

SEQ ID NO: 10 - DNA, 3741 bp, Solanum lycopersicum
  source 1..3741: mol_type = genomic DNA
atggctgaaaatgaaattgaggaaatgttagagcacctgagaaggatcaagagtggaggtgatctggattggctcgacatattgcgaattgaggaacttgaaatggtgctaagagtttttagaacctttacaaagtataatgatgttcttttgcctgattccttagtcgaactcacaaagagggccaaattgattggggaaatacttcaccggttgtttggtaggattccacataaatgtaaaactaaccttaatctagaaaggctagaatcacatttgttggaattctttcaaggtaatacggcaagtttaagtcacaattatgagttgaataattttgatctgtcgaaatatatggattgtctggaaaattttctaaatgatgtactgatgatgttcttgcaaaaggataggttcttccattccagagaacaacttgcaaaacatcgatcaataaaggaactgaaaattgttcaaaagaaaataagatttttgaaatacatatatgccacagagataaatggttatgtcgactatgagaagcaggaatgtttggagaatcgaattcagttcatgactaacactgtgggacaatattgtttggcagtattagattatgtcactgagggtaaacttaatgaagaaaatgacaactttagtaaacctccttacctattatcattgattgtgttagtggagctggaaatgaagaagatttttcatggtgaagtaaaggcttcaaagtttactcaatcaaaaactttcaaggacaagaaattaccaaaaggattttcacatcatctccacaatctgttgatgtatctcagaaacaaaaagctcgagaattttcctaataatatcgctgctcaaaatattgatgtggcaatagagttcttgttggttttccttgatgctgatgtgtcaaatcatgttattaatggtaactggttgaaaaaggtcttgttaaaggttggagctatagcgggtgatattctatatgtaattcaaaagcttcttcctagatcaataaacaaagatgaaactagcaacataagtctttgctcaatacagatattggagaagactaaagatctgaaggcacaagttgagacgtactacaaatccttaaaatttactccatctcagttccccacctttggtggattgagctttctgaattctcttttaaggaaactgaatgagatgtcgacatctaagtccggattaggtttcctgatgaaacctcttttagggaatttggagaaagagctatcatctcttacatccattttagagaaggagctctcatccattttccgtgatgtcgtgcaccatgaacataacattcctaaagatcttcagaggcgtaccatcaatttgtcatatgaggctgaggttgctattgattctattcttgctcagtataatgcttttttgcatattttttgctcacttcctacaattgtaaaagagatcaagcaaattaatgcagaggtgactgagatgtggtcagcggacattcctcttaatcctcactatgtggctgctccattaaaacatctgccggatcgacatagcaatcttgtaactgatgaggaggtagtgggttttgagaataaagcagaagaactaattgattatctgattagaggtacaaatgagctagacgttgtcccaattgtaggcatgggaggacaagggaaaacgacaattgctagaaagttgtacaataatgacattattgtttctcgctttgatgttcgagcatggtgcatcatttctcaaacgtataatcggagagagttattacaagatattttcagtcaagttacaggttccgacgacaatggagctacggttgatgttcttgccgacatgttgaggagaaaattaatgggaaagagatatctcattgtattggatgatatgtgggattgtatggtatgggatgacttaaggctttcttttccagatgatggaattagaagcagaatagtcgtaacaactcgacttgaagaagtgggtaagcaagtcaagtaccatactgatccttattctcttccattcctcacaacagaagagagttgccaattgttgcagaaaaaagtgtttcaaaaggaagattgcccgcctgaactacaagatgtgagtcaagcagtagcagaaaaatgcaaaggactgcccctagtggttgtcttggtagctggaataatcaaaaaaaggaaaatggaagaatcttggtggaatgaggtgaaagatgctttatttgactatcttgacagtgagttcgaagaatacagtctggcaactatgcagttgagttttgataacttaccccactgtttaaagccttgtcttctttatatgggaatgttttcggaggacgcaagaattccagcatctacattgataagtttatggattgctgaaggattcgtggagaacactgaatctgggagattaatggaagaggaagctgaaggttacttgatggatctcattagcagtaacttggtaatgctttcaaagagaacttataagggtagagtcaaatactgtcaggttcatgatgttgtgcatcacttttgcttggaaaagagtagagaagcaaagtttatgcttgcagtgaagggtcaatatatccattttcaaccttcggattggaagggaactcgagtgagcttcagttttagtgaagagctttccaagtttgcatctctggtctccaaaacacagaagcctttccatcaacacttgaggtcattgataacgaccaatcgagcaaaatctattaatgatattttctcctgtcagattagtgaattgcgacttcttaaagtcttggatttgagttcttatattgtggagtttttgtcgttagctacattcaaaccactaaatcagctgaagtacctcgcagttcaggcttttgaattctattttgatccaggatcacatcttccccatatagaaactttcattgtaatgaatcttccttattatgatatattgttaccagtgtctttttgggaaatgaaaaaattaaggcatgctcattttggtaaggctgaatttgacaagcaggggctctctgaaggatcctctaaattggaaaatttgaggatattaaagaatattgttggatttgatagggtggatgtgttatcaaggaggtgtcctaatcttcaacaacttcaaatcacatattttgggaataatgaagagcctttttgtcccaaattggagaatcttacccagcttcaacaacttcaactttcctttgcgcgtccccgcactctatccgggttacagttgccttcaaatttaaataagttggtacttgaaggaattcatataggatgtgttattcccttcattgcgggactaccaagcctggaatatctccagttacatgatgtgtgttttcctcaatcagaagagtggtgccttggagatatcacgttccataaacttaagttgttgaaactggtaaagttaaatatatcaaggtgggatgtctcagaggaatcatttccgttgcttgaaacactcgttataaagaagtgcattgacctagaggagattccacttagctttgctgatattccaacattggaacagattaaattgattgggtcctggaaagtatctctggaggattcagctgtgagaatgaaggaagaaatcaaagacactgaaggatgtgatcgtttacacctcgtcaaacaacactcagattga

SEQ ID NO: 11 - DNA, 3735 bp, Solanum sp.
  source 1..3735: mol_type = genomic DNA
tggctgaaaatgaaattgaggaaatgttagagcacctgaggaggatcaagagtggaggtgatctacattggttaaacatatttcaacttgagaaacttgaaatggtgctaagagtttttagaacctttacaaagtatcatgatgttcttttgcctgattccttagtcgaactcacaaagatggccaaattgactggggaagtacttcaccgggtgttgggtataattccacataaatgtaagcttaatctagaaaggctattatcacatttgttggaattctttcaaggtaatacggcaagtttaagtcacaattatgagttgaatgattttgatctgtcgaaatatatggattgtctggaaaattttctaaatgatttactgatgatgttctttcatgagggtagtttgtgccattccatagaacaacttttaatacatcgaacaataaaggaactgaaaattgttcaaaagaaaataagatttttgaaatacatatatgccacagagataaatggttatgtcgactatgagaagcaggaatgtttggagactcgaattcagttcatgactaacactgtgggacaatattgtttggccttattagattatgtcgctgggggtaaacttaataaagaaaatgacaactttagtaaacttccttacctattatcattgattgtgtttgtggagctggaaatgaagaagatttttcatggtgaagtaaaggcttcaaagtttactcaatcaaaaactttcaaggacaagaaattaccaaaaggattttcacatcatctccacaatctgttgatgtatctcagaaacaaaaagctcgagaattttcctaataatatcgctgctcaaaatattgatgtggcaatagagttcttgttggttttccttgatgctgatgtgtcaaatcatgttattaatggcaactggttgaaagaggttttgttaaaggttgtagctatagcgggtgatattctatatgtaattcaaaagcttcttcctagatcaataaacaaagatgaaactagcaacgtaagtctttgctcaatacagatattggagaagactaaagatctgaaggcacaagttgagacgtactacaaatccttaaaatttactccatctcagttccccacctttggtggattgagctttctggattctcttttaaggaaactgaatgagatgtcgacatctaagtccggattaggtttcctgatgaaacctcttttagggaatttggagaaagagctatcatctcttacatccattttagagaaggagctctcatccattttccgtgatgtcgtgcaccatgaacatagcattcctaaagatcttcagagacgtaccatcaatttggcatatgaggctgaggttgctattgattctattcttgctcagtataatgtttttttgcatattttttgctcacttcctacaattctaaaagagatcaagcaaattaatgcagaggtgactgagatgtggtcagcggatattcctcttaatcctcactatgtggctgctccattaaaacatctgcaagatcgacatagcaatcttgtaactgatgaggaggtagtgggttttgagaataaagcagaagaactaattgattatctgattagaggtacaaatgagctagacgttgtcccaattgtaggcatgggaggacaagggaaaacgacaattgctagaaagttgtacaataatgacattattgtttctcgctttgatgttcgagcatggtgcatcatttctcaaacgtataatcggagagagttattacaagatatttttagtcaagttacaggtgtcaacgacaatggagctacggttgacgatgttgccgacatgttgaggagaaaattaatgggaaagagatatctcattgtattggatgatatgtgggattgtatggtatgggatgacttaaggctttcctttccagattccggaaatagaagcagaatagtcgtaacaactcgacttgaagaagtgggtaagcaagtcaagtaccatactgatccttattctcttccattcctcacaacagaagagagttgccaattgttgcagaaaaaagtgtttcaaaaggacgattgcccgcctgaactacaagatgtgagtcaagcagtagcagaaaaatgcaaaggactgcccctagtggttgtcttggtagctggaataatcaaaaaaaggaaaatggaagaatcttggtggaatgaggtgaaagatgctttatttgactatattgacagtgagttcgaagaatatagtctggcgactatgcagttgagttttgataacttaccccactgtttaaagccttgtcttctttatatgggaatgttttcggaggacgcaagaattccagcatctacattgataagtttatggattgctgaaggattcgtggagaacactgaatctgggatattaatggaagaggcagctgaaggttacttgatggatctcattagcagtaacttggtaatgctttcaaagagaagttataagggtaaagtcaaatactgtcaggttcatgatgttgtgcatcacttttgcttggaaaagagtagagaagcaaagtttatgcttgcagtgaagggtcaatgtatccagtttcaaccttcggattggaagggtactcgagtgagcttcagttttagtgaagagctttccaagtttgcatctctggtctccaaaacacagaagcctttccatcaacacttgaggtcattgataacgaccaatcgagcaaaatctattaatgatattttctcctgtcagattagtgaattgcgacttcttaaagtcttggatttgagttcttatattgtggagtttttgtcgttagctacattcaaaccactaaatcagctgaagtacctcgcagttcaggcttttgaattctattttgatccaggatcacatcttccccatatagaaactttcattgtaatgaatcttccttattatgatatattgttaccagtgtctttttgggaaatgaaaaaattaaggcatgctcattttggtaaggctgaatttgacaagcaggggctctctgaaggatcctctaaattggaaaatttgaggatattaaagaatattgttggatttgatagggtggatgtgttatcaaggaggtgtcctaatcttcaacaacttcaaatcacatattttgggaataatgaagagcctttttgtcccaaattggagaatcttacccagcttcaacaacttcaactttcctttgcgcgtccccgcactctatccgggttacagttgccttcaaatttaaataagttggtacttgaaggaattcatataggatgtgttattcccttcattgcgggactaccaagcctggaatatctccagttacatgatgtgtgttttcctcaatcagaagagtggtgccttggagatatcacgttccataaacttaagttgttgaaactggtaaagttaaatatatcaaggtgggatgtctcagaggaatcatttccgttgcttgaaacactcgttataaagaagtgcattgacctagaggagattccacttagctttgctgatattccaacattggaacagattaaattgattgggtcctggaaagtatctctggaggattcagctgtgagaatgaaggaagaaatcaaagacactgaaggatgtgatcgtttacacctcgtcaaacaacactcagattgat

(19) United States
(12) Patent Application Publication
(10) Pub. No.: US 2026/0366038 A1
(43) Pub. Date: Nov. 19, 2026

(54) TOMATO PLANTS RESISTANT TO RESISTANCE-BREAKING TSWV STRAINS AND CORRESPONDING RESISTANCE GENES

(71) Applicant: VILMORIN & CIE, Paris (FR)

(72) Inventors: Céline PESCE, Davis, CA (US); John LINDBO, Woodland, CA (US); Kebede MULETA, Davis, CA (US)

(73) Assignee: VILMORIN & CIE, Paris (FR)

(21) Appl. No.: 19/144,083

(22) PCT Filed: Dec. 28, 2023
(86) PCT No.: PCT/EP2023/087919

§ 371 Date: Jun. 27, 2025

(30) Foreign Application Priority Data
Dec. 29, 2022 (EP) 22307060.8

(51) Int. Cl. C12N 15/82; C07K 14/415

(52) U.S. Cl. CPC C12N 15/8283; C07K 14/415

(57) ABSTRACT

A Sw-5b protein, which is a variant of the wild type protein, conferring recognition of the NSm protein of the Tomato Spotted Wilt virus (TSWV), and wherein said variant includes a variation at one or more of the following positions: 23, 26, 28, 29, 30, 75, 82, 83, 90, 134, 135, 136, 138, 139, 140, 143, 147, 148, 151, 203, 209, 214, 325, 351, 448, 522, 622 and 759 with respect to the sw-5b protein. The present invention also relates to genetic sequences encoding such a variant protein, and to plants, especially Solanum lycopersicum plants including in their genome said mutated gene conferring resistance to RB-TSWV. The invention is also directed to parts of these plants, as well as progeny, and to the use of these sequences for providing TSWV resistance.

The present invention relates to resistance in plants of Solanum lycopersicum, also known as Lycopersicum esculentum, to tospoviruses, especially to the Tomato Spotted Wilt virus (TSWV) including the resistance-breaking (RB)-Tomato Spotted Wilt viruses. More specifically, the present invention relates to tomato plants, cells, seed and fruits comprising a resistance gene and/or protein that leads to resistance to at least TSWV and RB-TSWV. According to the invention, the resistance gene conferring resistance to these tospoviruses is a variant or allele of the wild type sw-5b gene. The resistance gene can be present homozygously or heterozygously in the genome of a S. lycopersicum plant. The invention further relates to this resistance gene, parts thereof, to encoded polypeptides and proteins and to the use of these sequences and proteins to obtain resistant plants. The invention also relates to the seeds and progeny of such plants, to propagation material for obtaining such plants, and to different uses of these plants.

### BACKGROUND OF THE INVENTION

All cultivated and commercial forms of tomato belong to a species most frequently referred to as Lycopersicon esculentum Miller. Lycopersicon is a relatively small genus within the extremely large and diverse family Solanaceae which is considered to consist of around 90 genera, including pepper, tobacco and eggplant. The genus Lycopersicon has been divided into at least two subgenera, the esculentum complex which contains those species that can easily be crossed with the commercial tomato and the peruvianum complex which contains those species which are crossed with considerable difficulty. Due to its value as a crop, L. esculentum Miller has become widely disseminated all over the world.

Tomato is grown for its fruit, widely used as a fresh market or processed product. As a crop, tomato is grown commercially wherever environmental conditions permit the production of an economically viable yield. The majority of fresh market tomatoes are harvested by hand at vine ripe and mature green stage of ripeness. Fresh market tomatoes are available year round. Processing tomatoes are mostly mechanically harvested and used in many forms, as canned tomatoes, tomato juice, tomato sauce, puree, paste or even catsup.

Tomato is a normally simple diploid species with twelve pairs of differentiated chromosomes. However, polyploidy tomato is also part of the present invention. The cultivated tomato is self-fertile and almost exclusively self-pollinating. The tomato flowers are hermaphrodites. Commercial cultivars were initially open pollinated. As hybrid vigor has been identified in tomatoes, hybrids are replacing the open pollinated varieties by gaining more and more popularity amongst farmers with better yield and uniformity of plant characteristics. Due to its wide dissemination and high value, tomato has been intensively bred. This explains why such a wide array of tomato is now available. The shape may range from small to large, and there are cherry, plum, pear, blocky, round, and beefsteak types. Tomatoes may be grouped by the amount of time it takes for the plants to mature fruit for harvest and, in general the cultivars are considered to be early, midseason or late-maturing. Tomatoes can also be grouped by the plant's growth habit; determinate, semi-determinate or indeterminate. Determinate plants tend to grow their foliage first, then set flowers that mature into fruit if pollination is successful. All of the fruits tend to ripen on a plant at about the same time. Indeterminate tomatoes start out by growing some foliage, then continue to produce foliage and flowers throughout the growing season. These plants will tend to have tomato fruit in different stages of maturity at any given time. The semi-determinate tomatoes have a phenotype between determinate and indeterminate, they are typical determinate types except that grow larger than determinate varieties. More recent developments in tomato breeding have led to a wider array of fruit color. In addition to the standard red ripe color, tomatoes can be creamy white, lime green, pink, yellow, golden, orange or purple.

Hybrid commercial tomato seed can be produced by hand pollination. Pollen of the male parent is harvested and manually applied to the stigmatic surface of the female inbred. Prior to and after hand pollination, flowers are covered so that insects do not bring foreign pollen and create a mix or impurity. Flowers are tagged to identify pollinated fruit from which seed will be harvested.

A variety of pathogens affect the productivity of tomato plants, including virus, fungi, bacteria, nematodes and insects. Tomatoes are inter alia susceptible to many viruses and virus resistance is therefore of major agricultural importance.

Tospoviruses have a negative-sense, single-stranded RNA genome. The genome is divided into three segments termed S (2.9 kb), M (5.4 kb), and L (8.9 kb). The M and S RNA segments encode for proteins in ambisense direction. Tospoviruses take their name from the species Tomato Spotted wilt orthotospovirus (TSWV) which was discovers in Australia in the 20's.

Tospoviruses, and especially TSWV are vectorized inter alia by thrips, especially Frankliniella fusca and Frankliniella occidentalis.

TSWV has a very broad host range, including hundreds of species, either monocotyledonous and dicotyledonous plants, including tomato, pepper, lettuce, toabocco.

In tomatoes, the main symptoms of TSWV infection are Ringspot and Mosaic/Mottle following by curling, stunting, chlorosis and lesions on the plants.

For tomatoes, a resistance gene, namely sw-5b (or sw5b) gene (SEQ ID NO:10) encoding the CC-NBL-LRR protein Sw-5b, also known as Sw5b (SEQ ID NO:1) has however been identified by plants breeders over the years as providing resistance against TSWV as well as other tospoviruses, and TSWV resistant tomato varieties are nowadays available, comprising the sw-5b gene.

Sw-5b is a single dominant resistance gene, initially identified in S. peruvianum, and is to be found on chromosome 9.

Alternative genes have also been identified in other species infected by TSWV such as pepper.

Recently, TSWV strains capable of infecting sw5b-bearing tomatoes were however identified; the emergence of these new Resistance-Breaking TSWV, or RB-TSWV, is considered as a major global threat to tomato crop.

Multiple RB strains of TSWV have been characterized. The Resistance Breaking mutations map to the NSm gene of TSWV. In some commercial tomato growing operations more than 50% of the plants have become infected with RB-TSWV causing significant crop losses for growers (Crescenzi et al 2015). One of the most effective ways of combating RB-TSWV strain infections is the introduction of genetic resistance gene. Identification of a resistance gene against these new resistance-breaking TSWV strains has thus become important and urgent for tomato breeders.

The characterization of a putative resistance protein and gene, corresponding to a variant of the Sw-5b protein, has recently been disclosed (WO2015/090468; ISI Sementi). It however appears that these resistance protein and gene do not provide resistance, or sufficient resistance to the now circulating RB-TSWV strains (see example 3).

Plant NLR proteins have been extensively reviewed. NLRs are proteins that upon recognition of an ‘effector protein’ trigger a resistance response in plants. NLRs have been grouped into two major subclasses according to their N-terminal domain. The two major subclasses are CNLs and TNLs for proteins that contain either a coiled coil (C) or a toll/interleukin 1 receptor (TIR) domain, respectively at their N-terminus. In addition to the N-terminal domain NLRs also have a nucleotide binding (NB) domain and a Leucine Rich Repeat (LRR) domain. Each of the common domains (NB, LRR, CC and TIR) are proposed to play a role in activation of the NLR Protein (Wang et al. 2020).

In the case of the NLR protein Sw-5b, the TSWV movement protein NSm is the avirulent protein (elicitor) that is recognized by Sw-5b (Hallwass, et al., 2014; Peiro et al, 2014). Co-expression of Sw-5b and TSWV NSm has been shown to induce strong hypersensitive response (HR) in Nicotiana benthamiana and tomato leaves.

When the NSm protein of RB-TSWV isolates is used in this test, the co-expression of Sw-5b is no longer sufficient to induce HR.

Using a two-step artificial mutagenesis protocol, Huang et al, 2021 have recently disclosed two Sw-5b mutants, which are supposed to be effective against TSWV RB carrying the NSmC118Y or NSmT120N mutation. They have found inter alia that one mutation in LRR domain of Sw-5b was important, namely the R927A (by reference to the wildtype sequence represented by SEQ ID NO:1), but not sufficient to induce HR in the context of the whole protein. Further mutations in the N-terminal Solanaceae domain (SD) region were necessary, namely the simultaneous mutations L33P and K319E, in addition to the R927A mutation, were necessary to induce HR.

No tomato plants comprising these mutations were however disclosed, in order to confirm that these mutations are viable in solanum genetic background and effectively give rise to resistance to RB-TSWV strains without losing resistance to wildtype TSWV. Moreover, insofar as these mutants were artificially obtained, the corresponding mutant genes cannot be introgressed into commercial varieties.

There is thus an urgent need to identify improved resistance genes against several TSWV strains, including RB-TSWV strains, providing a broader, more effective resistance to TSWV, and which can easily be introgressed into tomato background.

The present inventors have unexpectedly found that a variant of the sw-5b gene, present in a wild accession not S. lycopersicum, can confer resistance to TSWV and RB-TSWV, and that this resistance gene is dominant. They have moreover demonstrated that this gene could be introgressed into S. lycopersicum background. They have also unexpectedly found that the variations with respect to the wild-type Sw-5b protein are not found in the LRR domain, which is generally considered as the interaction domain.

### SUMMARY

The present invention is directed to a Sw-5b protein, which is a variant from the wild type Sw-5b protein having SEQ ID NO:1, and which is able of recognizing the viral movement protein NSm of TSWV of wild-type strains, and also of resistance-breaking strains. Such a variant Sw-5b protein has at least 90% sequence identity to the wild type Sw-5b protein (represented by SEQ ID NO:1) and comprises at least one variation or mutation, including insertion and deletion, with respect to SEQ ID NO:1, which is preferably to be found at one or more of the following 28 positions: position 23, 26, 28, 29, 30, 75, 82, 83, 90, 134, 135, 136, 138, 139, 140, 143, 147, 148, 151, 203, 209, 214, 325, 351, 448, 522, 622 and 759.

The numbering of the amino acid is with respect to the wild type sequence represented by SEQ ID NO:1. The variation or mutation at one or more of these positions is preferably one or more of the following variations: substitution D23H, substitution D26N, substitution L28F, substitution R29Q, substitution I30L, substitution R75I, deletion T82DEL, deletion N83DEL, substitution E90L, substitution L134F, substitution Q135H, substitution K136E, substitution R138S, substitution F139L, substitution F140C, substitution R143I, substitution A147L, substitution K148I, substitution S151T, substitution V203L, substitution E209G, substitution E214K, substitution G325V, substitution I351V, substitution N448S, substitution P522Q, substitution V622D and substitution L759I.

Preferably, a variant Sw-5b protein of the invention comprises at least one variation or mutation with respect to SEQ ID NO:1:
in the 386-772 domain of the Sw-5b protein, wherein said variation or mutation is preferably at position 448, 522, 622 or 759, and more preferably is at least one of the following 4 variations with respect to SEQ ID NO:1: N448S; P522Q; V622D and L759I; and/or
in the 136-147 domain of the Sw-5b protein, wherein said variation or mutation is preferably at position 136, 138, 139, 140, 143, 147, and more preferably is at least one of the following variations with respect to SEQ ID NO:1: K136E, R138S, F139L, F140C, R143I and A147L.

Preferably, a variant Sw-5b protein of the invention comprises the following variations or mutations with respect to SEQ ID NO:1:
variations in the 386-772 domain of the Sw-5b protein, wherein said variations comprise at least the following 4 variations with respect to SEQ ID NO:1: N448S, P522Q, V622D and L759I; and/or
variations in the 136-147 domain of the Sw-5b protein, wherein said variations comprise at least 3 or at least 4 of the following variations: K136E, R138S, F139L, F140C, R143I and A147L.

The invention is also directed to different variants of Sw-5b protein, comprising additional variations or mutations, as well as nucleotide sequences encoding said proteins and polypeptides.

The invention also concerns a Resistance gene, encoding a mutant or allelic variant of the wild-type represented by SEQ ID NO:10, conferring to tomatoes resistance against several tosporviruses, and more specifically against TSWV, including strains which are considered as RB-TSWV strains.

The newly discovered resistance Sw-5b protein of the invention confers resistance against TSWV, including RB-TSWV, thanks to at least one mutation or variation in the wildtype protein, preferably at one of the 28 positions mentioned above, and more preferably thanks to at least one of the 28 substitutions or deletions mentioned, and even more preferably thanks to:
at least 1, 2, 3 or all of the four substitutions in the 386-772 domain of the wildtype Sw-5b protein, at position 448, 522, 622 and 759, namely the substitutions of the asparagine (N) 448 by a serine(S); proline (P) 522 by a glutamine (Q); the valine (V) 622 by an acid aspartic (D), and the leucine (L) 759 by an isoleucine (I); and/or
at least 1, 2, 3 or four substitutions in the 136-147 domain, chosen from the following substitutions: substitution of the lysine (K) 136 by a glutamic acid (E), the arginine (R) 138 by a serine(S), the phenylalanine (F) 139 by a leucine (L), the phenylalanine (F) 140 by a cysteine (C), the arginine (R) 143 by an isoleucine (I) and the alanine (A) 147 by a leucine (L); and/or
a combination of these substitutions.

Additional substitutions, and preferably substitutions in the 386-772 domain or in the 136-147 domain, as detailed below, may improve the resistance conferred by the substitutions disclosed above.

The present invention also provides plants, especially S. lycopersicum plants that display resistance to TSWV, including resistance to RB-TSWV, especially commercial plants, lines and hybrids, as well as methods that produce or identify plants, especially S. lycopersicum plants or populations (germplasm) that display resistance to TSWV and RB-TSWV. The present invention also discloses molecular genetic markers, linked to the newly discovered resistance gene. Plants obtained through the methods and uses of such molecular markers are also provided.

The invention also provides several methods, including methods for identifying TSWV and RB-TSWV resistant plants, methods for improving the yield of tomato production in an environment infested by different tospoviruses including TSWV, especially RB-TSWV, methods for protecting a tomato field from tospoviruses infestation including TSWV, especially RB-TSWV, and methods for identifying, detecting and/or selecting mutants or variants of the Sw-5b protein conferring resistance against at least TSWV and RB-TSWV.

### DEFINITIONS

The term “Resistance” is as defined by the ISF (International Seed Federation) Vegetable and Ornamental Crops Section for describing the reaction of plants to pests or pathogens, and abiotic stresses for the Vegetable Seed Industry. Specifically, by resistance, it is meant the ability of a plant variety to restrict the growth and development of a specified pest or pathogen and/or the damage they cause when compared to susceptible plant varieties under similar environmental conditions and pest or pathogen pressure. Resistant varieties may exhibit some disease symptoms or damage under heavy pest or pathogen pressure. Two levels of resistance are defined:

High Resistance: plants that highly restrict the growth and/or development of the specified pest and/or the damage it causes under normal pest pressure when compared to susceptible plants. These plants may, however, exhibit some symptoms or damage under heavy pest pressure.

Intermediate Resistance: plants that highly restrict the growth and/or development of the specified pest and/or the damage it causes but may exhibit a greater range of symptoms or damage compared to high resistance plants. Intermediate resistant plants will still show less severe symptoms or damage than susceptible plants when grown under similar environmental conditions and/or pest pressure.

The term “Tolerance” is normally used to describe the ability of a plant to endure abiotic stresses without serious consequences for growth, appearance and yield.

In the literature and patents, this term is however also used to indicate a phenotype of a plant wherein at least some of the disease-symptoms remain absent upon exposure of said plant to an infective dose of virus, whereby the presence of a systemic or local infection, virus multiplication, at least the presence of viral genomic sequences in cells of said plant and/or genomic integration thereof can be established, at least under some culture conditions. Tolerant plants are therefore resistant for symptom expression but symptomless carriers of the virus. Sometimes, viral sequences may be present or even multiply in plants without causing disease symptoms. It is to be understood that a tolerant plant, although it is infected by the virus, is generally able to restrict at least moderately the growth and development of the virus. For this reason, tolerant plants according to this definition are best characterized by Intermediate Resistant plants.

Symptoms of TSWV infection generally include Ringspot and Mosaic/Mottle following by curling, stunting, chlorosis and lesions.

Susceptibility: The inability of a plant to restrict the growth and development of a specified pest or pathogen; a susceptible plant displays the detrimental symptoms linked to the virus infection, namely the foliar damages and fruit damages in case of TSWV infection.

A S. lycopersicum plant susceptible to Tomato Spotted Wilt virus (TSWV) is for example the commercially available variety Tresor.

A resistance-breaking TSWV, or RB-TSWV, or RB-TSWV strain, is a mutant of the wild-type TSWV, capable of infecting plants comprising the wildtype resistance gene sw-5b, expressing the resistance Sw-5b protein. Such viruses are disclosed in Huang et al, 2021, and in WO2015/090468; ISI Sementi. A S. lycopersicum plant resistant to Tomato Spotted Wilt virus (TSWV) but susceptible to RB-TSWV is for example the commercially available variety YOKO.

As used herein, the term “offspring” or “progeny” refers to any plant resulting as progeny from a vegetative or sexual reproduction from one or more parent plants or descendants thereof. For instance, an offspring plant may be obtained by cloning or selfing of a parent plant or by crossing two parents plants and include selfings as well as the F1 or F2 or still further generations. An F1 is a first-generation offspring produced from parents at least one of which is used for the first time as donor of a trait, while offspring of second generation (F2) or subsequent generations (F3, F4, etc.) are specimens produced from selfings of F1's, F2's etc. An F1 may thus be (and usually is) a hybrid resulting from a cross between two true breeding parents (a true-breeding parent is homozygous for a trait), while an F2 may be (and usually is) an offspring resulting from self-pollination of said F1 hybrids.

As used herein, the term “cross”, “crossing”, “cross pollination” or “cross-breeding” refer to the process by which the pollen of one flower on one plant is applied (artificially or naturally) to the ovule (stigma) of a flower on another plant.

As used herein, the term “genotype” refers to the genetic makeup of an individual cell, cell culture, tissue, organism (e.g., a plant), or group of organisms.

As used herein, the term “grafting” is the operation by which a rootstock is grafted with a scion. The primary motive for grafting is to avoid damages by soil-born pest and pathogens when genetic or chemical approaches for disease management are not available. Grafting a susceptible scion onto a resistant rootstock can provide a resistant cultivar without the need to breed the resistance into the cultivar. In addition, grafting may enhance tolerance to abiotic stress, increase yield and result in more efficient water and nutrient uses.

As used herein, the term “heterozygote” refers to a diploid or polyploid individual cell or plant having different alleles (forms of a given gene, genetic determinant or sequences) present at least at one locus.

As used herein, the term “heterozygous” refers to the presence of different alleles (forms of a given gene, genetic determinant or sequences) at a particular locus.

As used herein, the term “homozygote” refers to an individual cell or plant having the same alleles at one or more loci on all homologous chromosomes.

As used herein, the term “homozygous” refers to the presence of identical alleles at one or more loci in homologous chromosomal segments.

As used herein, the term “hybrid” refers to any individual cell, tissue or plant resulting from a cross between parents that differ in one or more genes.

As used herein, the term “locus” (plural: “loci”) refers to any site that has been defined genetically, this can be a single position (nucleotide) or a chromosomal region. A locus may be a gene, a genetic determinant, a part of a gene, or a DNA sequence, and may be occupied by different sequences. A locus may also be defined by a SNP (Single Nucleotide Polymorphism), by several SNPs, or by two flanking SNPs.

As used herein, the term “rootstock” is the lower part of a plant capable of receiving a scion in a grafting process.

As used herein, the term “scion” is the higher part of a plant capable of being grafted onto a rootstock in a grafting process.

The invention encompasses plants of different ploidy levels, essentially diploid plants, but also triploid plants, tetraploid plants, etc.

### DETAILED DESCRIPTION OF THE INVENTION

The present inventors have identified variants of the Sw-5b protein, such that said protein comprising variations or mutations with respect to the wildtype protein Sw-5b, corresponding to SEQ ID NO:1, recognizes and/or binds the Movement Proteins (MP) of several tospoviruses, including the NSm protein of at least TSWV (Tomato Spotted Wilt virus) and preferably also at least some of the Resistance-Breaking TSWV (RB-TSWV), most preferably all RB-TSWV.

The present invention is thus directed to variants of the Sw-5b protein, namely variants of SEQ ID NO:1, SEQ ID NO:1 corresponding to a sequence of the wild-type Sw-5b protein. Said variants according to the invention have at least 90% sequence identity with SEQ ID NO:1, at the amino acid level, and said variants confer, if expressed in a tomato plant, the capacity to recognize and/or bind at least the Movement Protein NSm of TSWV, and preferably also of at least some or all of the RB-TSWV.

By way of contrast, the wild-type Sw-5b protein, i.e. SEQ ID NO:1 or similar sequences, confers the capacity to bind NSm of only wild type TSWV, but does not confer significant binding to or recognition of NSm of RB-TSWV. Variants of the Sw-5b protein of the invention are interchangeably referred to as Sw-5b proteins, Sw-5b protein variants, mutants or alleles of the invention.

A Sw-5b protein is said to recognize the NSm protein of TSWV if said protein binds, directly or indirectly, but preferably directly, said TSWV NSm; the direct or indirect binding may be at the level of, or involves one or more domains of the Sw-5b protein, such as the SD domain, or the LRR domain, or may involve the whole protein. Such a recognition can be tested by the assay disclosed in the examples, in N. benthamiana.

Such a Sw-5b protein is also referred to as a resistance protein of the invention in the following.

A Sw-5b protein variant or allele according to the invention comprises variation(s) in its amino acid sequence with respect to the wild type sequence as represented by SEQ ID NO:1, i.e. one or more variations, the variation(s) being preferably found at one or more of the following 28 positions within the protein: position 23, 26, 28, 29, 30, 75, 82, 83, 90, 134, 135, 136, 138, 139, 140, 143, 147, 148, 151, 203, 209, 214, 325, 351, 448, 522, 622 and 759. Said positions are unexpectedly not in the LRR domain of the Sw-5b protein.

Preferably, a Sw-5b protein according to the invention, which is a variant of the wild-type sequence, comprises at least one, but preferably at least 2, 3, or 4 variations with respect to SEQ ID NO:1, which are to be found at said 28 positions mentioned above. In some embodiments, a protein of the invention comprises variations with respect to SEQ ID NO:1 at more than 4 of said 28 positions, preferably at at least 5 of said positions, preferably at least 8 or 10 of said positions. A protein of the invention may also comprise one or more variations with respect to SEQ ID NO:1 at other positions.

The variation or mutation at one or more of these 28 positions is preferably one or more of the following variations: substitution D23H, substitution D26N, substitution L28F, substitution R29Q, substitution I30L, substitution R75I, deletion T82DEL, deletion N83DEL, substitution E90L, substitution L134F, substitution Q135H, substitution K136E, substitution R138S, substitution F139L, substitution F140C, substitution R143I, substitution A147L, substitution K148I, substitution S151T, substitution V203L, substitution E209G, substitution E214K, substitution G325V, substitution I351V, substitution N448S, substitution P522Q, substitution V622D and substitution L759I, with respect to SEQ ID NO:1. According to other embodiments, a protein of the invention comprises at least 2, 3 or 4 of the above-mentioned variations, either substitutions or deletions. According to some embodiments, a protein of the invention comprises more than 4 of these variations, with respect to SEQ ID NO:1, preferably at least 5, 8 or 10 of said variations or even more.

Preferably, a variant Sw-5b protein of the invention comprises at least one variation or mutation with respect to SEQ ID NO:1:
in the 386-772 domain of the Sw-5b protein, wherein said variation or mutation is preferably at position 448, 522, 622 or 759, and more preferably is at least one of the following 4 variations with respect to SEQ ID NO:1: N448S, P522Q, V622D and L759I; and/or
in the 136-147 domain of the Sw-5b protein, wherein said variation or mutation is preferably at position 136, 138, 139, 140, 143 or 147, and more preferably is at least one of the following variations/mutations with respect to SEQ ID NO:1: K136E, R138S, F139L, F140C, R143I and A147L.

According to a preferred embodiment, a Sw-5b protein variant or allele according to the invention comprises thus variations in its amino acid sequence with respect to the wildtype sequence as represented by SEQ ID NO:1, the variations being found either in the 386-772 domain, or in the 136-147 domain, or in both domains. Preferably, there is no variation in the LRR domain.

### The 386-772 Domain of Sw-5b

The 386-772 domain (adopting the numbering based on SEQ ID NO:1) has indeed been demonstrated in WO2015/090468 has being involved in the recognition of TSWV, modifications in this region providing resistance to RB-TSWV according to WO2015/090468. The corresponding sequence of the mutant Sw-5b2 protein described in WO2015/090468 is illustrated in FIG. 3 and corresponds to SEQ ID NO:5.

The present inventors have however noted that, contrary to the teaching of WO2015/090468, plants comprising the Sw-5b2 protein are not significatively more resistant to the tested RB-TSWV isolates than plants considered as susceptible to RB-TSWV comprising the wildtype Sw-5b protein. They have however identified other mutations or variations, in this region, with respect to the wild type sequence, which provide resistance to RB-TSWV, either on their own or in combination with additional variations, such as some of those disclosed in WO2015/090468.

Particularly, a variant of the wildtype Sw-5b protein, conferring resistance to TSWV, including RB-TSWV, comprises at least one variation or mutation, preferably 2, 3 or 4, in said 386-772 domain. Preferred positions in said domain are positions 448, 522, 622 and 759. Preferred substitutions at these positions are the following ones:
substitution of the asparagine (N) 448 by a serine(S);
substitution of the proline (P) 522 by a glutamine (Q);
substitution of the valine (V) 622 by an acid aspartic (D), and
substitution of the leucine (L) 759 by an isoleucine (I).

In a preferred embodiment, a variant of the wildtype Sw-5b protein, conferring resistance to TSWV, including RB-TSWV, comprises at least the following 4 variations in the 386-772 domain, at positions 448, 522, 622 and 759:
substitution of the asparagine (N) 448 by a serine(S);
substitution of the proline (P) 522 by a glutamine (Q);
substitution of the valine (V) 622 by an acid aspartic (D) and
substitution of the leucine (L) 759 by an isoleucine (I).

A Sw-5b protein according to the invention, which is a variant of the wildtype sequence SEQ ID NO:1, preferably comprises at least one, and preferably, 2, 3 or all of said 4 variations, referred to in the following as primary variations of the 386-772 domain, potentially in combination with other variations, although variants comprising only 3 or less, of said variations are also encompassed by the present invention.

According to alternative embodiments, a Sw-5b protein according to the invention also comprises additional modifications with respect to the wildtype sequence, especially one of the following variations, referred to as secondary variations:
substitution of the alanine (A) 477 by a valine (V),
substitution of the valine (V) 489 by a leucine (L),
substitution of the acid aspartic (D) 659 by a serine(S) and
substitution of the glutamic acid (E) 707 by acid aspartic (D).

Preferably a Sw-5b protein according to the invention comprises at least one of these additional variations, preferably two or three of these additional variations, and most preferably these four additional variations.

Whereas some of these variations may sometimes be found in plants susceptible to RB-TSWV, they are thought to improve the resistance to RB-TSWV when present in combination with one or more of the first four variations, namely in combination with one or more of N448S, P522Q, V622D and L759I and preferably in combination with said four variations.

A preferred Sw-5b protein according to the invention thus comprises the N448S, P522Q, V622D and L759I variations, and at least one, and preferably 2, 3 or the 4 A477V, V489L, D659S and E707D variations.

In another embodiment, the present invention also concerns a Sw-5b protein, comprising one or more of the following variations with respect to the wildtype sequence SEQ ID NO:1, said variations being referred to as tertiary variations of the 386-772 domain, in addition to the primary substitutions:
the substitution of the asparagine (N) 393 by an aspartic acid (D);
the substitution of the serine(S) 461 by an alanine (A);
the substitution of the aspartic acid (D) 614 by an asparagine (N);
the substitution of the leucine (L) 623 by a valine (V) and
the substitution of the isoleucine (I) 661 by an asparagine (N).

Preferably, a Sw-5b protein according to the invention comprises at least 1, 2, 3, 4 or the 5 above-mentioned additional variations. These variations are also found in the sw-2b2 protein disclosed in WO2015/090468. As mentioned previously, these variations, contrary to the disclosure of WO2015/090468 do not provide, on their own, resistance to RB-TSWV. They are however suspected to increase the resistance to RB-TSWV, in a Sw-5b protein variant already comprising at least one or more of the four primary variations N448S, P522Q, V622D and L759I, and preferably comprising the four primary variations.

In a preferred embodiment, a Sw-5b protein according to the invention comprises the four primary variations, in combination with at least one, and preferably at least 2, or 3, or all 4 secondary mutations, and also at least one of the tertiary variations, and preferably more than one of the tertiary variations. Preferably, a Sw-5b protein according to the invention, which is a variant of the SEQ ID NO:1, comprises with respect to this sequence all primary, secondary and tertiary variations, but one, either a secondary or a tertiary variation, or alternatively comprises all variations, except two variations, which are either two secondary variations, or two tertiary variations, or one of each.

In another embodiment, in addition of the secondary and/or tertiary variations of the 386-772 domain, a Sw-5b protein according to the invention may also comprise a variation at position 613 of the protein, with respect to SEQ ID NO:1, substituting the serine(S) at this position by a valine or a cysteine, preferably a valine.

According to a preferred embodiment, a Sw-5b protein according to the invention thus comprises at least the following 13 variations, with respect to a wild type Sw-5b protein as represented by SEQ ID NO: 1: N393D, N448S, S461A, A477V, V489L, P522Q, D659S, D614N, V622D, L623V, I661N, E707D and L759I; or at least 10, 11 or 12 of these variations, provided it comprises N448S; P522Q; V622D and L759I. According to another embodiment, a Sw-5b protein according to the invention comprises the 14 following variations with respect to SEQ ID NO:1: N393D; N448S; S461A, A477V; V489L; P522Q; D659S, S613V, D614N, V622D; L623V, 1661N, E707D and L759. The corresponding 386-772 domain of such a variant is represented by SEQ ID NO:4.

In a preferred embodiment, a Sw-5b protein of the invention comprises the 386-772 domain having SEQ ID NO:4. The protein may or may not comprise additional variations in the other domains of Sw-5b. Irrespective of these variations, the percentage of sequence identity, at the whole protein level, is preferably at least 90% between the protein of the invention and SEQ ID NO:1.

### The 136-147 Domain of Sw-5b

The 136-147 domain (adopting the numbering based on SEQ ID NO:1) is thought to be directly involved in the interaction between the NSm protein of wildtype TSWV and the Sw-5b protein, indeed, according to Li et al., 2019 and Seong et al., 2022, the interaction site of Sw-5 with NSm of the wildtype strains of TSWV was allegedly mapped between positions 136 and 147 (Nter SD domain).

The present inventors have identified mutations or variations, with respect to the wild type sequence, in this region, and in the extended region 134-151, which provide resistance to RB-TSWV, either on their own or in combination with additional variations, such as some of those disclosed in WO2015/090468.

In one embodiment, a variant of the wild type Sw-5b protein, conferring resistance to TSWV, including RB-TSWV, comprises at least one variation or mutation, preferably 2, 3 or 4, in the 134-151 domain, and more preferably in the 136-147 domain. Preferred positions in said domain are positions 134, 135, 136, 138, 139, 140, 143, 147, 148 and 152, and more preferably positions 136, 138, 139, 140, 143 and 147. Preferred substitutions at these positions are substitution L134F, substitution Q135H, substitution K136E, substitution R138S, substitution F139L, substitution F140C, substitution R143I, substitution A147L, substitution K148I and substitution S151T.

Particularly, a variant of the wild type Sw-5b protein, conferring resistance to TSWV, including RB-TSWV, comprises at least 1, 2, 3 or preferably at least 4 of the following primary variations in the 136-147 domain:
substitution of the lysine (K) 136 by a glutamic acid (E);
substitution of the arginine (R) 138 by a serine(S);
substitution of the phenylalanine (F) 139 by a leucine (L);
substitution of the phenylalanine (F) 140 by a cysteine (C);
substitution of the arginine (R) 143 by an isoleucine (I); and
substitution of the alanine (A) 147 by a leucine (L).

Preferably, Sw-5b protein according to the invention comprises at least 3, at least 4, preferably 5 or more preferably the 6 above-mentioned primary variations. If so, a preferred Sw-5b protein according to the invention thus comprises the K136E, R138S, F139L, F140C, R143I and A147L variations with respect to the wildtype Sw-5b sequence represented by SEQ ID NO:1.

In an alternative embodiment, a Sw-5b protein of the invention also comprises a mutation at position 137, namely a substitution of the aspartic acid (D) by a glycine (G).

The corresponding 136-147 domain of a variant comprising said variation at position 137 in addition to the 6 primary variations in the 136-147 domain is represented by SEQ ID NO:7.

According to other preferred embodiments, a Sw-5b protein according to the invention also comprises additional secondary variations, in the very near proximity of the 136-147 domain. A preferred protein thus comprises at least one of the following variations:
substitution of the leucine (L) 134 by a phenylalanine,
substitution of the glutamine (Q) 135 by an histidine (H),
substitution of the lysine (K) 148 by an isoleucine (I) and
substitution of the serine(S) 151 by a threonine (T).

A Sw-5b protein according to the invention preferably comprises at least two or three of these additional secondary variations, in addition to the at least 3 or at least 4 primary variations disclosed above; and preferably these 4 additional secondary variations, in addition to the at least 4 primary variations.

Particularly preferred Sw-5b proteins of the invention thus comprise one of the following combinations of variations in the 134-151 domain:
(1) K136E, R138S, F139L, F140C, R143I, A147L and L134F;
(2) K136E, R138S, F139L, F140C, R143I, A147L and Q135H,
(3) K136E, R138S, F139L, F140C, R143I, A147L and K148I and
(4) K136E, R138S, F139L, F140C, R143I, A147L and S151T.

A Sw-5b protein according to the invention may also comprise at least 3, at least 4, preferably 5 or more of the following variations: L134F, Q135H, K136E, R138S, F139L, F140C, R143I, A147L, K148I and S151T with respect to the wildtype Sw-5b sequence represented by SEQ ID NO:1.

According to another embodiment, a sw-5b protein of the invention comprises a 134-151 domain combining all the variations mentioned above in this region (primary, secondary and position 137); such a region is as set forth in SEQ ID NO:9.

In a preferred embodiment, a Sw-5b protein of the invention comprises the 136-147 or 134-151 domains having SEQ ID NO:7 and 9 respectively. The protein may or may not comprise additional variations in the other domains of Sw-5b. Preferably, the protein also comprises the 386-772 domain having SEQ ID NO:4. Irrespective of these variations, the percentage of sequence identity, at the whole protein level, is preferably at least 90% between the protein of the invention and SEQ ID NO:1.

### Further Modifications

A Sw-5b protein according to the invention thus comprises variations with respect to the wild type sequence, preferably at one or more of the 28 positions identified, and/or comprises variations either in the 136-147 domain, wherein the variations are as disclosed above, or in the 386-772 domain, wherein the variations are also as disclosed previously, or in both domains.

In addition to these variations preferably present in one of the two domains or in both domains, the protein of the invention may also advantageously comprise variations in other segments of the protein, which may increase the interaction between the protein and the NSm protein of TSWV, and in particular of RB-TSWV.

Such advantageous additional variations are preferably the deletion of the amino acid 82 (threonine) and/or amino acid 83 (asparagine) in the variant according to the invention. In such a case, a Sw-5b protein according to the invention comprises one and preferably two amino acids less than the wildtype sequence, namely amino acid 82 or 83, or both 82 and 83. According to another independent embodiment, this deletion may give rise to resistance to RB-TSWV even in the absence of the other variations mentioned above in the 386-772 or 136-147 domains or 134-151 domain.

A further additional variation, which is advantageous in the context of the present invention is the substitution at position 319 of the lysine (K) by a glutamic acid (E). Such a substitution has indeed been demonstrated, in Huang et al, as participating in the TSWV resistance.

A Sw-5b protein according to the invention has preferably at least 90% sequence identity, at the amino acid level, with a wildtype protein as represented by SEQ ID NO:1, and comprises at least the variations described above, preferably in the 136-147 domain or in the 386-772 domain.

Sequence identity between two amino acid sequences is as defined usually in the domain of the invention. A suitable program for defining sequence identity is for example Clustal Omega.

According to preferred embodiments, the Sw-5b protein of the invention comprises at least 92% sequence identity at the protein level, over the whole sequence of the protein; preferably at least 94% sequence identity over the whole sequence.

According to a particularly preferred embodiment, the sequence of a Sw-5b protein according to the invention comprises at least 95% sequence identity with a wildtype sequence represented by SEQ ID NO:1.

Regarding other variations or mutations, present in a Sw-5b protein of the invention, in addition to those defined at the 28 identified positions, and/or in the 136-147 domain and/or in the 386-772 domain, they are preferably conservative amino-acids substitutions, preferably preserving the 3D structure of the protein.

Namely a basic amino acid like lysine or arginine is preferably substituted by another basic amino acid; an acidic amino acid like aspartic acid or glutamic acid is preferably substituted by another acidic amino acid. A small apolar amino acid like glycine, alanine, proline, cysteine or valine is preferably substituted by another small apolar amino acid. A large apolar amino acid like leucine, isoleucine, phenylalanine, methionine or tryptophan is preferably substituted by another large apolar amino acid. A small polar amino acid like serine or threonine is preferably substituted by another small polar amino acid. A large polar amino acid like asparagine or glutamine is preferably substituted by another large polar amino acid. An aromatic amino acid like tyrosine, phenylalanine or tryptophan is preferably substituted aby another aromatic amino acid.

Such a protein however advantageously does not comprise variation or mutation at the position corresponding to position 33 in SEQ ID NO:1, namely the protein comprises a leucine at the position corresponding to L33 in SEQ ID NO:1. Similarly, a protein according to the invention preferably does not comprises a mutation at the position corresponding to position 927 in SEQ IDNO:1, namely it comprises an arginine (R) at the position corresponding to R927 in SEQ ID NO:1. More generally, a protein according to the invention preferably does not comprises a mutation or variation in the LRR domain.

A particularly preferred Sw-5b protein according to the invention, which is thus a variant of the wild type sequence SEQ ID NO:1, is SEQ ID NO:3, or a sequence sharing at least 99% sequence identity with SEQ ID NO:3, especially at those positions which have been described above.

As mentioned, the Sw-5b protein of the invention recognizes, and in fact interacts, with the viral movement protein NSm of TSWV, including the movement protein NSm of RB-TSWV strains, especially of RB-TSWV strains characterized by a mutation in the C118 position of this virus, e.g. the C118Y or C118F variant of TSWV which are known as a RB-TSWV, and the strains characterized by mutations at the 137 and 153 positions, such as the V137I and V153I mutations, or by a mutation at position 120, e.g. the T120N mutation. Those strains are known as RB-TSWV, or have been demonstrated as such by the inventors.

A Sw-5b protein according to the invention can be distinguished from a wild-type Sw-5b by its capacity to interact with the NSm protein of these RB-TSWV mutants, especially the C118Y and C118F mutants, and the mutant comprising the mutations V137I and V153I. The experimental section discloses simple assays to demonstrate the interaction between a Sw-5b protein, either wild type or variant, with a NSm protein of a TSWV, either wild type or RB-TSWV. The ability of a Sw-5b protein to confer the capacity to recognize and/or bind the Movement Protein NSm of a TSWV can indeed be tested easily with the transient expression assay disclosed in the examples, namely by transient expression in N. benthamiana of a Sw-5b protein comprising the variations to be tested, in presence of the NSm protein of TSWV, and by screening for hypersensitive response (HR).

It is noted in this regard that detection of a hypersensitive response in a transient expression assay in a surrogate plant like N. benthamiana equates detection of resistance in tomato. There is indeed a correlation between the ability of a Sw-5b protein to trigger a robust HR response in N. benthamiana leaves (in the presence of a tospovirus NSm protein) and virus resistance in tomato. This correlation has been confirmed inter alia with another tomato virus, namely ToBRFV. Indeed, in WO 2022/117884, it has been demonstrated that a hypersensitive response in the surrogate assay with transient expression in N. benthamiana of a Tm-2-2 gene variant and the movement protein of ToBRFV is indicative of the ToBRFV resistance in tomato plants comprising the Tm-2-2 gene variant.

The variant protein according to the invention not only provides resistance to TSWV, including RB-TSWV, but also provides resistance to different tospoviruses, as does wildtype Sw-5b protein. The variations according to the invention thus preferably represent a gain of a new resistance, namely resistance against RB-TSWV, without any loss, i.e. all the resistances provided by the wildtype Sw-5b protein are maintained.

Such a variant protein; once expressed in the corresponding plant, can provide resistance to tospoviruses and more specifically to TSWV and RB-TSWV not only in S. lycopersicum plants, but also in other species and gender, especially commercial plants, which are susceptible to TSWV. In particular, a variant protein according to the invention may provide resistance to TSWV infection in tomato, peppers, potato and/or lettuce, including to resistance-breaking TSWV strains comprising the C118Y or C118F mutation and strains comprising the V137I and V153I mutations.

The present invention is also directed to a nucleotide sequence encoding a Sw-5b protein, or protein variant, as defined above. In view of the degeneracy of the genetic code, very different nucleotide sequences can be envisaged, coding for a protein of the invention, namely coding for a variant of SEQ ID NO:1, characterized essentially by the variations at one or more of the 28 identified positions, and/or in the 136-147 domain and/or in the 386-772 domain, and/or in both domains.

A suitable nucleotide sequence is for example a sequence corresponding to SEQ ID NO:11, namely the sequence encoding the specific variant of the Sw-5b protein identified by the inventors.

The present invention also concerns sequences deriving from SEQ ID NO:10, corresponding to the wildtype sequence of sw-5b gene, having at least 70% sequence identity with said sequence, preferably at least 75%, at least 80%, at least 85%, at least 90% or at least 95% sequence identity. Irrespective of the percentage of sequence identity with SEQ ID NO:10, a sequence according to the invention encodes a Sw-5b protein of the invention as defined above. Given the degeneracy of the genetic code, a sequence encoding a Sw-5b variant of the invention may nevertheless have less than 70% sequence identity with SEQ ID NO:10, and is also in the scope of the present invention.

A nucleotide sequence according to the invention encompasses at least DNA, single-stranded DNA, RNA, double stranded RNA, and mixture of DNA and RNA, as well as complementary sequence thereof. The sequence can be isolated or not.

The invention according to another aspect also relates to a Resistance gene, encoding a Sw-5b protein which is a variant, mutant or allele of the Sw-5b protein, conferring to plants, especially tomatoes, but also pepper, potato and/or lettuce resistance against at least TSWV, and preferably also against at least one or more RB-TSWV. Preferably, such a resistance gene confers resistance against at least RB-TSWV comprising a C118Y or C118F mutation, or comprising the V137I and V153I mutations.

The mutant, variant or allele of the Sw-5b protein is a variant of the wild type Sw-5b protein as disclosed above, comprising at least the disclosed variations at one or more of the 28 positions of SEQ ID NO:1 identified by the inventors, and preferably in the 386-772 domain, or in the 136-147 domain, or in both. The resistance gene encoding the Sw-5b protein of the invention as defined previously, is also referred to a resistant allele of sw-5b gene, or the sw-5b allele of the invention.

The newly discovered resistance protein and the resistance gene encoding said protein, confer resistance against TSWV, and in particular against some or all of the TSWV variants known as RB-TSWV, thanks to the substitutions described in the preceding, in one or both of the domains mentioned, or at one or more of the identified positions.

According to still another aspect, the invention is also directed to a nucleic acid construct comprising a sequence encoding a Sw-5b protein according to the invention, i.e. a variant of the wildtype sequence represented by SEQ ID NO:1, or comprising a resistance gene according to the invention.

Such a sequence, encoding a Sw-5b protein of the invention, or comprising a resistance gene is interchangeably referred to as a nucleotide sequence or resistance gene, or variant sw-5b gene or sw-5b allele of the invention, in the following.

Such a sequence encoding the protein, polypeptide or variant of interest is preferably under the control of a promoter, which is a constitutive or an inducible promoter. Preferably, the promoter is a promoter which is active in plant cells. According to an embodiment, the promoter is not the wild-type promoter of sw-5b gene. Preferably the promoter for the sw-5b variant gene is however the native promoter of sw-5b gene.

A nucleic acid construct according to the invention can thus be a vector, a plasmid or a T-DNA plasmid. Presence of a construct of the invention in a cell thus may give rise, under appropriate conditions, to the expression of a protein, polypeptide Sw-5b variant or Resistance protein according to the invention and as defined above.

The invention also encompasses expression vector or construct suitable for expression of a polypeptide, protein, Sw-5b variant or resistance protein according to the invention, preferably expression in a plant cell.

According to a further embodiment, the invention is also directed to the use of a sequence as defined, encoding a Sw-5b allele of the invention, or a construct comprising such a sequence for conferring resistance against TSWV to a S. lycopersicum plant or for obtaining transgenic S. lycopersicum plants resistant against TSWV, including RB-TSWV, at least those comprising the C118Y or C118F mutation, or the V137I and V153I mutations. Indeed, as demonstrated in the example, the sw-5b variant recognizes the TSWV NSm and also the RB-TSWV NSm, thus triggering a HR response associated with TSWV resistance.

According to a further aspect, the invention also concerns a cell comprising a nucleotide sequence or resistance gene according to the invention or a DNA construct as disclosed above, or a resistance protein or Sw-5b protein variant of the invention.

The cell is preferably a plant cell, preferably from the Solanaceae family, for example from the Solanum genus, and even more preferably a cell of S. lycopersicum plant, or a cell of the Capsicum or Nicotiana genus, preferably a cell of N. tabacum, N. benthamiana, or Capsicum annuum.

The cell expresses, transiently or constitutively, a Sw-5b protein according to the invention, which is thus a variant of the wild type Sw-5b protein, as described in the foregoing.

According to a preferred embodiment, the cell comprises, in its genome and preferably in its nuclear genome, a nucleotide sequence or resistance gene or DNA or nucleic acid construct according to the present invention and described in the foregoing. The presence of these sequences confers the phenotype of interest, namely expression of a protein interacting with at least the TSWV NSm protein, including of RB-TSWV, triggering resistance, or HR response in suitable conditions. The presence of these sequences can be revealed by any techniques well known to the skilled reader, based on the sequence.

Particularly preferred types of cells are cells of the Solanum, Nicotiana or Capsicum genus, and more preferably S. lycopersicum cells.

Cells according to the invention can be any type of cell, especially of S. lycopersicum cell, inter alia an isolated cell and/or a cell capable of regenerating a whole plant, especially a S. lycopersicum plant bearing the nucleotide sequence or resistance gene of the invention. A cell can thus be a regenerable cell or a non regenerable cell.

The nucleotide sequence or resistance gene of interest can be present homozygously or heterozygously in a cell of the invention. Preferably, a cell according to the invention comprises the resistance gene, or the nucleotide sequence as defined above, at the heterozygous state. The Sw-5b gene is indeed recognized as being a dominant one. The presence of the resistance gene of the invention may however be preferred, as providing a greater resistance under certain conditions of TSWV infection, or RB-TSWV infection.

The present invention is also directed to a tissue culture of non-regenerable or regenerable cells as defined above according to the present invention; preferably, the regenerable cells are derived from embryos, protoplasts, meristematic cells, callus, pollen, leaves, anthers, stems, petioles, roots, root tips, fruits, seeds, flowers, cotyledons, and/or hypocotyls, and the cells contain the nucleotide sequences or resistance gene in their genome conferring the resistance against at least TSWV. Preferably, such sequences or resistance gene also provide resistance against one or more RB-TSWV, and preferably against al least the RB-TSWV strains comprising the C118Y or C118F mutation or the V137I and V153I mutations in the TSWV genome.

The invention is also directed to any plant part, especially a S. lycopersicum plant part, particularly seeds, explants, reproductive material, scion, cutting, seed, fruit, root, rootstock, pollen, ovule, embryo, protoplast, leaf, anther, stem, petiole or flowers, wherein said plant part comprises at least one cell as described above.

The invention also provides a protoplast comprising the nucleotide sequences or resistance gene of the invention.

According to another aspect, the invention is directed to a plant, and more preferably to a S. lycopersicum or a Capsicum annuum plant, comprising in its genome a nucleotide sequence or a resistance gene as defined above, encoding a Sw-5b protein of the invention, which is a variant of the wildtype Sw-5b protein as described in the preceding. Such a nucleotide sequence or resistance gene thus encodes for a variant of the Sw-5b protein, comprising at least one variation with respect to SEQ ID NO:1, which is preferably to be found at one or more of the following 28 positions: position 23, 26, 28, 29, 30, 75, 82, 83, 90, 134, 135, 136, 138, 139, 140, 143, 147, 148, 151, 203, 209, 214, 325, 351, 448, 522, 622 and 759. The variation or mutation at one or more of these positions is preferably one or more of the following variations: substitution D23H, substitution D26N, substitution L28F, substitution R29Q, substitution I30L, substitution R75I, deletion T82DEL, deletion N83DEL, substitution E90L, substitution L134F, substitution Q135H, substitution K136E, substitution R138S, substitution F139L, substitution F140C, substitution R143I, substitution A147L, substitution K148I, substitution S151T, substitution V203L, substitution E209G, substitution E214K, substitution G325V, substitution I351V, substitution N448S, substitution P522Q, substitution V622D and substitution L759I.

In a preferred embodiment, a nucleotide sequence or resistance gene encodes for a variant of the Sw-5b protein, comprising
(1) at least 1, 2, 3 or preferably at least four variations in the 136-147 domain of SEQ ID NO:1, which are chosen amongst K136E, R138S, F139L, F140C, R143I and A147L or
(2) one, two, three or preferably the four variations N448S, P522Q, V622D and L759I with respect to SEQ ID NO:1, or
(3) a combination of the variations (1) and (2);
and conferring resistance against TSWV infection, preferably including RB-TSWV infection, especially at least by RB-TSWV strains or isolates comprising the C118Y or C118F mutation, or the V137I and V153I mutations.

Preferably the nucleotide sequence or resistance gene encodes a variant of the Sw-5b protein further comprising other mutations or variations as described in the preceding, inter alia the dinucleotide deletion at positions 82 and 83 of the wildtype sequence, and/or variations surrounding the 136-147 domain, namely at positions 134-151 with respect to SEQ ID NO:1.

As already mentioned in the previous sections and applicable to all aspects of the invention, a particularly preferred nucleotide sequence or resistance gene encodes a variant of the Sw-5b protein corresponding or comprising SEQ ID NO:3, or a protein having at least 95% or preferably at least 99% sequence identity with SEQ ID NO:3. A suitable nucleotide sequence encoding such a protein is SEQ ID NO:11.

The resistance gene or nucleotide sequence as defined may be present homozygously or heterozygously in a plant of the invention. It can provide resistance to TSWV, including RB-TSWV, at the heterozygous state. These sequences can also be present as multiple copies.

Said resistance gene or nucleotide sequence of the invention is preferably present in the genomic DNA of the plant of the invention, such as a S. lycopersicum plant.

The invention thus also encompasses a S. lycopersicum plant resistant against TSWV, comprising an allele of the Sw-5b gene encoding a Sw-5b protein having at least 90% sequence identity to the wild type Sw-5b protein (SEQ ID NO:1) and comprising at least 1, 2, preferably at least 3 and more preferably at least 4 of the following variations with respect to SEQ ID NO:1: K136E, R138S, F139L, F140C, R143I and A147L; and preferably at least 5 of these variations, and even more preferably these 6 variations. One or more additional variations in the near proximity, especially at positions 134, 135, 148 and 151 may also be present.

According to another embodiment, the invention also encompasses a S. lycopersicum plant resistant against TSWV, comprising an allele of the Sw-5b gene encoding a Sw-5b protein having at least 90% sequence identity to the wild type Sw-5b protein (SEQ ID NO:1) and comprising at least 1, 2, 3 and more preferably all of the following 4 variations with respect to SEQ ID NO:1: N448S, P522Q, V622D and L759I; and preferably at least some of the additional variations, in the 386-772 domain already disclosed in previous sections.

Such plants exhibit resistance to TSWV, especially to RB-TSWV, such as the RB-TSWV comprising mutations at position 118, and/or at positions 137 and 153.

A plant of the invention, especially a S. lycopersicum plant may advantageously combine variations in the 136-147 domain and in the 386-772 domain.

The resistance gene or nucleotide sequence of the invention may also encode a Sw-5b protein comprising variations with respect to SEQ ID NO:1 which are at one or more of the 28 identified positions, but not in the 136-147, 134-151 or 386-772 domains, either in combination with variations in one or more of these domains, or not.

The resistance gene or nucleotide sequence of the invention is preferably stably present in the nuclear genome of the cells of the plant. It can be either stably integrated into the nuclear genome, for example after transformation, or it can result from a mutagenesis process such as Tilling. The presence of these sequences conferring resistance against TSWV, including RB-TSWV, may also result from introgression from a resistant parent. These sequences conferring resistance against tospoviruses, including at least TSWV and RB-TSWV, are preferably but not necessarily, to be found on chromosome 9, at the locus of the sw-5b gene. Other positions in the genome, for example resulting from random integration, are also suitable and encompassed by the present invention.

According to a specific embodiment, this resistance gene or nucleotide sequence of the invention is incorporated into a plant, especially a S. lycopersicum plant by introgression or homologous recombination, and in such a case, the nucleotide sequence or resistance gene is normally to be found on chromosome 9 of a S. lycopersicum plant.

The resistance phenotype can be tested and scored as described in the experimental section, by natural infection through thrips or by artificial mechanical inoculation.

The invention is also directed to tissue of a plant of the invention; the tissue can be an undifferentiated tissue, or a differentiated tissue. Such a tissue comprises one or more cells comprising the resistance sw-5b gene of the invention.

The invention is also directed to propagation material, capable of producing a resistant plant according to the invention, especially a resistant tomato plant, comprising the resistance gene or mutated sw-5b gene as defined above, encoding a Sw-5b protein of the invention. The invention is particularly directed to seeds of such a resistant plant, comprising the resistance gene, especially S. lycopersicum seeds and seed which can be grown into a S. lycopersicum plant according to the invention. Such plants are indeed particularly valuable as they are resistant to TSWV infection, and inter alia infection by resistance-breaking train of TSWV.

The seeds of such S. lycopersicum are preferably coated or pelleted with individual or combined active species such as plant nutrients, enhancing microorganisms, or products for disinfecting the environment of the seeds and plants. Such species and chemicals may be a product that promotes the growth of plants, for example hormones, or that increases their resistance to environmental stresses, for example defense stimulators, or that stabilizes the pH of the substrate and its immediate surroundings, or alternatively a nutrient.

They may also be a product for protecting against agents that are unfavorable toward the growth of young plants, including herein viruses and pathogenic microorganisms, for example a fungicidal, bactericidal, hematicidal, insecticidal or herbicidal product, which acts by contact, ingestion or gaseous diffusion; it is, for example, any suitable essential oil, for example extract of thyme. All these products reinforce the resistance reactions of the plant, and/or disinfect or regulate the environment of said plant. They may also be a live biological material, for example a nonpathogenic microorganism, for example at least one fungus, or a bacterium, or a virus, if necessary with a medium ensuring its viability; and this microorganism, for example of the pseudomonas, bacillus, trichoderma, clonostachys, fusarium, rhizoctonia, etc. type stimulates the growth of the plant, or protects it against pathogens.

A plant, cell or seed of the invention may be heterozygous or homozygous for the resistance gene or sw-5b allele of the invention conferring TSWV resistance. The present invention thus also encompasses plant, cell or seed having heterozygously in their genome the resistance gene of the invention as defined above.

Preferably, a S. lycopersicum plant according to the invention is a commercial plant or line. Such a commercial plant or line preferably also exhibits one or more of the following additional features: ToMV/TMV resistance (Tm-2 or Tm-22 resistance gene) nematode resistance trait (Mi-1 or Mi-j), Fusarium resistance, Verticillium resistance, and/or TYLCV resistance.

Other resistances or tolerances are also envisaged according to the invention.

Moreover, the commercial S. lycopersicum plant of the invention preferably gives rise to fruits in suitable conditions, which are at least 10 grams, preferably 25 grams at full maturity, preferably at least 100 g at full maturity and or even more preferably at least 150 g or at least 200 g at full maturity. The number of fruits per plant is moreover essentially unaffected by the presence of the resistance gene of the invention, i.e. the productivity of a plant according to the invention is not inferior by more than 20% to a plant having the same genotype but devoid of said resistance gene.

According to still another embodiment, a S. lycopersicum plant of the invention is a determinate, indeterminate or semi-indeterminate plant, or seed or cell thereof, i.e. corresponding to determinate, indeterminate or semi-indeterminate growth habit.

By determinate, it is meant tomato plants which tend to grow their foliage first, then set flowers that mature into fruit if pollination is successful. All of the fruits tend to ripen on a plant at about the same time. Indeterminate tomatoes start out by growing some foliage, then continue to produce foliage and flowers throughout the growing season. These plants will tend to have tomato fruit in different stages of maturity at any given time. The semi-determinate tomatoes have a phenotype between determinate and indeterminate, they are typical determinate types except that grow larger than determinate varieties.

According to still another embodiment, a plant of the invention is used as a scion or as a rootstock in a grafting process. Grafting is a process that has been used for many years in crops such as cucurbitacea, but only more recently for tomato. Grafting may be used to provide a certain level of resistance to telluric pathogens such as Phytophthora or to certain nematodes. Grating is therefore intended to prevent contact between the plant or variety to be cultivated and the infested soil. The variety of interest used as the graft or scion, optionally an F1 hybrid, is grafted onto the resistant plant used as the rootstock. The resistant rootstock remains healthy and provides, from the soils, the normal supply for the graft that it isolates from the diseases.

As detailed above, the invention is more specifically directed to S. lycopersicum plants, exhibiting the TSWV resistance, including resistance to RB-TSWV, as well as to seeds giving rise to those plants, and cells of these plants or seeds, or other plant parts, comprising the resistance gene in their genome, and to progeny of such a plant of the invention comprising said resistance gene.

Progeny encompasses the first, the second, and all further descendants from a cross with a plant according to the invention, wherein a cross comprises a cross with itself or a cross with another plant. Preferred progenies correspond to the first and second generation.

It is noted that the seeds or plants of the invention may be obtained by different processes, and are not exclusively obtained by means of an essentially biological process. The resistance gene encoding the Sw-5b protein of the invention can indeed be introduced, incorporated or obtained “in cellulo” by different techniques. Plants, cells or seeds according to the invention may thus be transgenic, or non-transgenic, they are preferably obtained by technical processes which are not essentially biological processes, as detailed in other sections of this description and in the examples. According to a preferred embodiment, the plants, cells or seeds according to the invention are not exclusively obtained by means of an essentially biological process.

A resistance gene, encoding the Sw-5b protein variant as disclosed, may advantageously be obtained by gene editing techniques, base-editing or prime editing techniques, such a mutagenesis, especially targeted mutagenesis such a TILLING, or by other gene editing techniques such as CRISPR/Cas system, or by custom-made endonucleases, or by base-editing or prime editing with Cas9, Cas12a or other Cas proteins.

These techniques are well known to the skilled reader.

In specific embodiments of the invention, the variations in the sw-5b gene are induced by means of genetic engineering. The genetic engineering means which can be used include the use of all such techniques called New Breeding Techniques which are various new technologies developed and/or used to create new characteristics in plants through genetic variation, the aim being targeted mutagenesis, targeted introduction of new genes or gene silencing (RdDM). Example of such new breeding techniques are targeted sequence changes facilitated through the use of Zinc finger nuclease (ZFN) technology (ZFN-1, ZFN-2 and ZFN-3, see U.S. Pat. No. 9,145,565), Oligonucleotide directed mutagenesis (ODM), Cisgenesis and intragenesis, Grafting (on GM rootstock), Reverse breeding, Agro-infiltration (agro-infiltration “sensu stricto”, agro-inoculation, floral dip), Transcription Activator-Like Effector Nucleases (TALENs, see U.S. Pat. Nos. 8,586,363 and 9,181,535), the CRISPR/Cas system (see U.S. Pat. Nos. 8,697,359; 8,771,945; 8,795,965; 8,865,406; 8,871,445; 8,889,356; 8,895,308; 8,906,616; 8,932,814; 8,945,839; 8,993,233; and 8,999,641), engineered meganuclease re-engineered homing endonucleases, DNA guided genome editing (Gao et al., Nature Biotechnology (2016)), and Synthetic genomics. A major part of targeted genome editing, another designation for New Breeding Techniques, is the applications to induce a DNA double strand break (DSB) at a selected location in the genome where the modification is intended. Directed repair of the DSB allows for targeted genome editing. Such applications can be utilized to generate mutations (e.g., targeted mutations or precise native gene editing) as well as precise insertion of genes (e.g., cisgenes, intragenes, or transgenes). The applications leading to mutations are often identified as site-directed nuclease (SDN) technology, such as SDN1, SDN2 and SDN3. For SDN1, the outcome is a targeted, non-specific genetic deletion mutation: the position of the DNA DSB is precisely selected, but the DNA repair by the host cell is random and results in small nucleotide deletions, additions or substitutions. For SDN2, a SDN is used to generate a targeted DSB and a DNA repair template (a short DNA sequence identical to the targeted DSB DNA sequence except for one or a few nucleotide changes) is used to repair the DSB: this results in a targeted and predetermined point mutation in the desired gene of interest. As to the SDN3, the SDN is used along with a DNA repair template that contains new DNA sequence (e.g. gene). The outcome of the technology would be the integration of that DNA sequence into the plant genome. The most likely application illustrating the use of SDN3 would be the insertion of cisgenic, intragenic, or transgenic expression cassettes at a selected genome location. A complete description of each of these techniques can be found in the report made by the Joint Research Center (JRC) Institute for Prospective Technological Studies of the European Commission in 2011 and titled “New plant breeding techniques—State-of-the-art and prospects for commercial development”.

A resistance gene as defined may also be introduced into a plant, cell or seed of the invention by transformation, especially Agrobacterium transformation, thus producing transgenic plants, cells or seeds.

The application is directed to plants, seed or cells comprising the resistance gene or sw-5b allele of the invention as already defined irrespective of the mode of provision of these sequences; and thus is indifferently directed to transgenic and non-transgenic plants.

In an embodiment, the plant, cell or seed of the invention are transgenic plant, cell or seed, or genetically modified plant, cell or seed, in order to incorporate the resistance gene or sw-5b allele of the invention.

In a further aspect, the invention is also directed to different methods for obtaining, breeding or producing plants, especially S. lycopersicum plant resistant against TSWV, including RB-TSWV.

The invention thus encompasses methods of producing a plant, especially S. lycopersicum plant resistant to TSWV, and preferably also resistant to RB-TSWV comprising the following steps:
a) treating M0 seeds of a plant, preferably a tomato plant to be modified with a mutagenic agent, preferably comprising the wild type sw-5b gene coding for a wild type Sw-5b protein, to obtain M1 seeds;
b) growing plants from the thus obtained M1 seeds to obtain M1 plants;
c) producing M2 seeds by self-fertilisation of M1 plants; and
d) optionally repeating step b) and c) n times to obtain M1+n seeds.

The M1 or M2 seeds are grown into plants and submitted to TSW and/or RB-TSWV infection or to screening to identify variations in the sw-5b gene.

In this method, the M1 seeds of step a) can be obtained via chemical mutagenesis such as EMS mutagenesis or by other chemical mutagenic agents or physical means, such as irradiation, which is for example selected ionizing radiations (X-Ray, gamma rays, alpha particles . . . ), heavy-ion beam irradiation, ultraviolet radiations, radioactive decay or fast neutrons irradiation.

Another method for producing a S. lycopersicum plant resistant against TSWV, preferably including RB-TSWV, comprises the introduction into a plant already comprising a sw-5b gene on chromosome 9, of mutations into said sw-5b gene, in order to create a mutated sw-5b gene according to the invention, namely encoding a Sw-5b protein according to the invention, displaying the variation with respect to SEQ ID NO:1 already detailed.

When the starting material is a S. lycopersicum plant comprising a sw-5b gene, a preferred method may advantageously comprise the introduction of mutations in said sw-5b gene, preferably by mutagenesis, by TILLING or by genome editing, base-editing or prime editing, in particular by mutagenesis induced by a physical agent or a chemical agent, inter alia by a technique selected from ethyl methanesulfonate (EMS) mutagenesis, N-methyl-N-nitrosourea (MNU) mutagenesis, or Sodium Azide (NaN3, SA) mutagenesis, oligonucleotide directed mutagenesis (ODM), Zinc finger nuclease (ZFN) technology, Transcription Activator-Like Effector Nucleases (TALENs), the CRISPR/Cas system, Cas9, Cas12a, or other Cas proteins, engineered meganuclease, re-engineered homing endonucleases and DNA guided genome editing, wherein said mutations give rise to at least the substitutions in the protein encoded by the sw-5b gene which have already been detailed, at one or more of the 28 identified positions, preferably in the 136-147 domain or in the 386-772 domain, or in both domains, especially.
(1) at least mutations giving rise to 1, 2, 3 and preferably all of the following four mutations in the protein N448S, P522Q, V622D and L759I; or
(2) at least mutations giving rise to 1, 2, 3 and preferably 4 or more of the following mutations in the protein: K136E, R138S, F139L, F140C, R143I and A147L, or
(3) mutations giving rise to a combination of (1) and (2).

The method may comprise the introduction of further mutations in the sw-5b gene, giving rise to additional mutations in the Sw-5b protein, as mentioned previously.

Additional mutations may also be introduced, provided the recognition of the TSWV and RB-TSWV NSm protein is not lost.

The invention also encompasses different methods for obtaining transgenic plants according to the invention, especially transgenic S. lycopersicum plants resistant against TSWV, including RB-TSWV strains, by introducing a resistance gene according to the invention. These methods may comprise the following steps:
Obtaining a DNA construct as defined in a preceding aspect of the invention, i.e. comprising a resistance sw-5b gene or allele, encoding a Sw-5b protein according to the invention,
Introducing said construct into a cell, especially into a S. lycopersicum cell,
Regenerating a transgenic plant and
Optionally propagating the obtained plant.

According to another aspect, the present invention is also directed to the use of a tomato seed or plant of the invention, preferably comprising homozygously the resistance gene of the invention, as a breeding partner in a breeding program for obtaining S. lycopersicum plants having the TSWV resistance phenotype, and preferably also RB-TSWV resistance. Indeed, such a breeding partner harbors homozygously in its genome the resistance gene conferring the phenotype of interest. By crossing this plant with a tomato plant, especially a line, it is thus possible to transfer the resistance gene of the present invention conferring the desired phenotype, to the progeny. A plant according to the invention can thus be used as a breeding partner for introgressing the resistance gene into a S. lycopersicum plant or germplasm. Although a plant or seed bearing heterozygously the resistant gene of interest, can also be used as a breeding partner as detailed above, the segregation of the phenotype is likely to render the breeding program more complex.

The invention thus also concerns a method for breeding a S. lycopersicum plant resistant against TSWV and preferably also to RB-TSWV, including the strains having mutations at the C118 position, and at the V137 and V153 positions, comprising:
(a) Crossing a S. lycopersicum plant comprising a resistance gene according to the invention with an initial S. lycopersicum plant devoid of said resistance gene but potentially comprising a wildtype sw-5b gene,
(b) Selecting in the progeny thus obtained, a plant bearing the resistance gene of the invention,
(c) Optionally self-pollinating one or several times the plant obtained at step (b) and selecting in the progeny thus obtained a plant bearing the resistance gene.

The selection can be made by any appropriate means well known to the skilled person, inter alia by using markers specific to the resistance gene or mutated gene.

The invention is also directed to a method of producing a S. lycopersicum plant resistant to TSWV, including mutant strains comprising mutations such that they are considered as RB-TSWV, comprising obtaining a part of a plant according to the invention, thus comprising a resistance gene as already defined, and vegetatively propagating said plant part to generate a plant from said plant part.

In all the methods and processes according to the invention, the S. lycopersicum plant is determinate, indeterminate or semi-determinate.

As already disclosed, the tomato plants according to the invention are preferably also resistant to nematodes, TMV, ToMV, TYLCV, Fusarium and/or Verticillium.

The present invention is also directed to a S. lycopersicum plant and seed obtained or obtainable by any of the methods and processes disclosed above. Such a plant is indeed a S. lycopersicum plant expressing a Sw-5b variant of the invention conferring resistance against tospoviruses, especially against TSW and RB-TSWV.

According to still another aspect, the invention is also directed to a method for genotyping a plant, preferably a S. lycopersicum plant or tomato germplasm, for the presence of a resistance gene or mutated sw-5b gene according to the invention associated with resistance against TSWV infection, including RB-TSWV, wherein the method comprises the determination or detection in the genome of the tested plant of a nucleic acid comprising or corresponding to at least one of the following variations, substitutions or deletions: substitution D23H, substitution D26N, substitution L28F, substitution R29Q, substitution I30L, substitution R75I, deletion T82DEL, deletion N83DEL, substitution E90L, substitution L134F, substitution Q135H, substitution K136E, substitution R138S, substitution F139L, substitution F140C, substitution R143I, substitution A147L, substitution K148I, substitution S151T, substitution V203L, substitution E209G, substitution E214K, substitution G325V, substitution 1351V, substitution N448S, substitution P522Q, substitution V622D and substitution L759I.

Preferably, the method comprises the determination or detection in the genome of the tested plant of a nucleic acid comprising or corresponding to K136E, R138S, F139L, F140C, R143I, A147L, N448S, P522Q, V622D and L759I mutations or substitutions in the sw-5b gene encoding the Sw-5b protein. Preferably, the method comprises the step of identifying in a sample of the plant to be tested specific sequences associated with at least one of these variations, which are specific to the sw-5b allele identified by the inventors. According to a preferred embodiment, the method comprises the determination or detection in the genome of the tested plant of
(1) at least mutations giving rise to 1, 2, 3 and preferably all of the following four mutations in the protein: N448S, P522Q, V622D and L759I; or
(2) at least mutations giving rise to 1, 2, 3 and preferably 4 or more of the following mutations in the protein: K136E, R138S, F139L, F140C, R143I and A147L, or
(3) mutations giving rise to a combination of (1) and (2), conferring resistance to TSWV, including RB-TSWV.

Similarly, the invention is also directed to a method for identifying, detecting and/or selecting S. lycopersicum plants resistant to RB-TSWV in plants known to be resistant to TSWV, said method comprising the detection of a mutant allele of the sw-5b gene in the genome of said plants, wherein said mutant allele comprises at least one mutation or variation chosen from the mutations giving rise to the following substitutions or deletions: D23H, D26N, L28F, R29Q, I30L, R75I, T82DEL, N83DEL, E90L, L134F, Q135H, K136E, R138S, F139L, F140C, R143I, A147L, K148I, S151T, V203L, E209G, E214K, G325V, 1351V, N448S, P522Q, V622D and L759I, with respect to SEQ ID NO:1.

According to a preferred embodiment, the mutant allele comprises at least one mutation or variation chosen from the mutations giving rise to a K136E, R138S, F139L, F140C, R143I, A147L, N448S, P522Q, V622D and L759I mutations or variations in the encoded protein, with respect to SEQ ID NO:1. In this regard, molecular markers are also envisaged in the context of the present invention, specifically designed to detect or select S. lycopersicum plants resistant to resistance-breaking strains of TSWV, amongst plants resistant to TSWV; said markers are able to discriminate at least one of the following variations in the sequence encoded by SEQ ID NO:10: D23H, D26N, L28F, R29Q, I30L, R75I, T82DEL, N83DEL, E90L, L134F, Q135H, K136E, R138S, F139L, F140C, R143I, A147L, K148I, S151T, V203L, E209G, E214K, G325V, I351V, N448S, P522Q, V622D and L759I; and preferably at least one of the following variations: K136E; R138S; F139L; F140C; R143I; A147L, N448S; P522Q; V622D and L759I, and L759I. Suitable markers are generally less than 50 nucleotide long, and are at least 95% identical to the corresponding portion of SEQ ID NO:10. Design of suitable markers or primers is routine for a skilled reader. Preferably, in order to design a KASP assay, three primers are used, two allele-specific forward primers, and one common reverse primer. The two allele-specific primers differ only at the 3′ end, one being specific to the “resistant” allele, and the other one to the “susceptible” or wild type allele. The allele specific primers are advantageously labelled with a fluorescent dye.

Other techniques are also well known to the skilled reader, in order to design markers distinguishing between two alleles of a given polymorphism.

In view of the ability of the resistant plants of the invention to restrict the damages caused by different tospoviruses infection, including TSWV and RB-TSWV infection, they are advantageously grown in an environment infested or likely to be infested or infected by TSW and RB-TSWV; in these conditions, the resistant plants of the invention produce more marketable tomatoes than susceptible plants. The invention is thus also directed to a method for improving the yield of tomato plants in an environment infested by TSWV and RB-TSWV, comprising growing tomato plants comprising in their genome the resistance gene as defined according to the previous aspects of the invention, and conferring to said plants resistance to TSWV as well as to RB-TSWV.

Preferably, the method comprises a first step of choosing or selecting a tomato plant comprising said resistance gene of interest. The method can also be defined as a method of increasing the productivity of a tomato field, tunnel or glasshouse, or as a method of reducing the intensity or number of chemical or fungicide applications in the production of tomatoes.

The invention is also directed to a method for reducing the loss on tomato production in condition of TSWV infestation or infection, and more specifically in condition of RB-TSWV infestation, comprising growing a tomato plant as defined above.

These methods are particularly valuable for a population of tomato plants, either in a field, in tunnels or in glasshouses.

Alternatively, said methods for improving the yield or reducing the loss on tomato production may comprise a first step of identifying tomato plants resistant to TSWV including RB-TSWV, and comprising in their genome the resistance gene of the invention, that confers to said plants at least TSWV resistance, including resistance to RB-TSWV, and then growing said resistant plants in an environment infested or likely to be infested by the virus.

The resistant plants of the invention are also able to restrict the growth of TSWV, and ore specifically of RB-TSWV, thus limiting the infection of further plants and the propagation of the virus, especially of those mutant viruses breaking the resistance provided by the wildtype sw-5b protein. Accordingly, the invention is also directed to a method of protecting a field, tunnel or glasshouse, or any other type of plantation, from RB-TSWV infection, or of at least limiting the level of infection by RB-TSWV of said field, tunnel or glasshouse or of limiting the spread of RB-TSWV in a field, tunnel or glasshouse, especially in a tomato field. Such a method preferably comprises the step of growing a resistant plant of the invention, i.e. a plant comprising in its genome the resistance gene, conferring to said plant RB-TSWV resistance, especially resistance against the mutant strains comprising mutation at C118 and at V137 and V153 positions.

The invention also concerns the use of a plant resistant to RB-TSWV for controlling RB-TSWV infection or infestation in a field, tunnel or glasshouse, or other plantation; such a plant is a plant of the invention, comprising in its genome the resistance gene as defined above. This use or method is also a method for disinfecting a field, tunnel or glasshouse by decreasing its viral population.

In still a further aspect, the invention also relates to a method of producing tomatoes comprising:
a) growing a S. lycopersicum plant of the invention, comprising the resistance gene as defined previously;
b) allowing said plant to set fruit; and
c) harvesting fruit of said plant, preferably at maturity and/or before maturity.

All the preferred embodiments regarding the resistance gene are already disclosed in the context of the previous aspects of the invention. The method may advantageously comprise a further step of processing said tomatoes into a tomato processed food.

The invention also concerns a process for the production of tomatoes in a transgenic tomato plant, comprising introducing into a S. lycopersicum plant a nucleic acid molecule coding for a Sw-5b protein according to the invention, having the substitution in the 386-772 or/and in the 136-147 domain, or having a variation as described, at one or more of the 28 identified positions. The process may further comprise a step of regenerating a transgenic plant, and allowing the plant to set fruit. The process may also comprise a step of harvesting fruit(s) of said transgenic plant.

### Sequences:

SEQ ID No:1: amino acid sequence of Sw-5b protein of Mospomor (wildtype);
SEQ ID No:2: amino acid sequence of domain 386-772 of SEQ ID NO:1;
SEQ ID No:3: amino acid sequence of Sw-5b protein of Line 111 (resistant);
SEQ ID No:4: partial sequence of SEQ ID NO:3 corresponding to domain 386-772 of SEQ ID NO:1;
SEQ ID No:5: amino acid sequence of sw-5b2 (corresponding to domain 386-772 of SEQ IDNO:1).
SEQ ID No:6: amino acid sequence of domain 136-147 of SEQ ID NO:1.
SEQ ID No:7: partial sequence of SEQ ID NO:3 corresponding to domain 136-147 of SEQ ID NO:1
SEQ ID No:8: amino acid sequence of domain 134-151 of SEQ ID NO:1.
SEQ ID No:9: partial sequence of SEQ ID NO:3 corresponding to domain 134-151 of SEQ ID NO:1.
SEQ ID No:10: nucleotide sequence of sw-5b gene of Mospomor (wildtype)
SEQ ID No:11: nucleotide sequence of sw-5b gene of line 111 (resistant)

### LEGEND OF THE FIGURES

FIG. 1: this graph illustrates the percentage of plants comprising virus, as detected by 4 weeks after inoculation. ELISA test has been performed on each plant. Two RB-TSWV strains (one strain, strain 1, having the same properties and mutations as strains circulating inter alia in South America, and one strain, strain 2, having the same properties and mutations as strains circulating inter alia in Italy) and one TSWV strain have been tested on 3 genotypes: Line homozygous for sw5b (sw5b line), a line without sw5b (no sw5b line), and the wild (non-S. lycopersicum) accession 111.

FIG. 2: this graph illustrates the presence of the virus in the inoculated plants after inoculation, evidenced by ELISA test. The RB-TSWV strain 1 has been tested on 3 genotypes: Line homozygous for sw5b (sw5b line), a line without sw5b (no sw5b line), and two different hybrid, comprising the sw5b2 variant allele of sw5b.

FIG. 3: this figure is an alignment of sequences of different Sw-5b proteins. Sw5b amino acids sequences from the wild accession Line 111 (SEQ ID NO:3), the wild-type sw5b Mospomor line (Resistant to TSWV strains only) (SEQ ID NO:1), and the sequence from WO2015/090468 (Sw5b2, which corresponds to a partial sequence of the full-length sw-5b, SEQ ID NO:5). The 6 amino acids in line 111 and in Sw5b2 which are identical in these two lines and different from the wild-type sw5b Mospomor line are underlined; the 8 amino acids of Line 111 which are different from those of Sw5b2 and wild-type sw-5b corresponding to SEQ ID NO:1 are shown in italics and lower case. The 28 substitutions of the wild accession Line 111, which are found neither in the wild-type Sw-5b sequence, nor in the Sw5b2 variants, nor in other susceptible variants, are double-underlined. The LLR domain is in italics, upper case.

FIG. 4: this photograph illustrates the HR reaction when Sw5b and NSm protein of TSWV are co-expressed or expressed individually. The expression of Tm22 and the movement protein of TMV is used as positive control of HR.

FIG. 5: this photograph illustrates the HR reaction. The picture was done 6 days post infiltration. The spots on the left half of the leaf (L) have been infiltrated with Sw5b from Mospormor (wild type) and the spots of the right half of the leaf (R) have been infiltrated with the Sw5b from Accession 111. Spots 1 (L1, R1) correspond to the NSm wild-type protein (TSWV strain), the spots 2 (L2, R2) correspond to NSm variant with a mutation C118Y, spots 3 (L3, R3) correspond to a NSm variant not identified as a RB-TSWV, and the spots 4 (L4, R4) correspond to NSm variant with mutations V137I and V153I.

FIG. 6: this graph illustrates the presence of the virus in the inoculated plants after inoculation, evidenced by ELISA test. The tested plants are the F1, 3 different BC1F1 and susceptible controls EP7 and Mospomor.

### EXAMPLES

The inventors screened more than wild 50 Solanum accessions, from their own proprietary lines, different from S. lycopersicum, for resistance to RB-TSWV strains, after identification of mutations in these strains (Example 2) rendering them resistant to sw-5b resistance gene, using a mechanical inoculation test. The tested wild accessions are from species known to comprise resistance, although these resistances are frequently difficult to introgress, such as S. habrochaites.

One such wild accession, line 111, showed good levels of resistance to 2 different RB-TSWV strains or isolates (Example 3).

To better understand this resistance the sw-5b gene of line 111 was cloned (pJL 491 clone) and sequenced and compared to the wild-type sw-5b allele (pJL 488 clone) currently used in the breeding programs, and not providing resistance to RB-TSWV strains (Example 4).

Results indicated there was 95.3% identify between the two protein alleles of Sw-5b, at the amino acid level.

The inventors thus hypothesized that the improved resistance is due to an improved ability of the newly-identified Sw-5b allele to respond to NSm proteins from RB-TSWV isolates.

Hallwass et al., have demonstrated that transient expression of Sw-5b (wild-type) protein and an NSm (wild-type) protein in N. benthamiana leaves result in a hypersensitive response (HR). This HR is triggered by binding of the NSm protein by the Sw-5b protein. Using this system, in example 5, the inventors have demonstrated that Sw-5b protein of line 111 detects NSm protein variants from RB-TSWV strains and triggers a robust HR response. In contrast, the Sw-5b (wt) protein does not recognize the NSm protein from these same RB-TSWV strains.

The inventors then transferred the sw-5b gene from the wild accession 111 to commercial tomato lines (example 6).

### Example 1: Material and Methods

### Viral Strains:

Two RB-TSWV strains (one strain having the same properties and mutations as strains circulating inter alia in South America, named “RB-TSWV strain 1” in the following and one strain having the same properties and mutations as strains circulating inter alia in Italy, named “RB-TSWV strain 2” in the following) have been tested. The isolate of RB-TSWV strain 1 comprises the C118F mutation and the V137I mutation. The isolate of RB-TSWV strain 2 comprises at least the C118Y mutation.

### Viral Inoculation:

The RB-TSWV strains or isolates were multiplied in leaves of susceptible plants. The symptomatic leaves are harvested and stored in −80 °C.

The optimal results are obtained by using Datura plant as maintainer. Fresh datura plants are mechanically inoculated with frozen ground tissue infected with the RB-TSWV strain. The new infected datura leaves correspond to the 3^{rd} passage inoculum. Tomato plants are inoculated with this the fresh inoculated Datura leaves. This corresponds to the 3^{rd} passage (1^{st} passage: original material frozen in-80 °C, 2^{nd} passage: Datura leaves, 3^{rd} passage: Tomato plants). A second inoculation could be done.

Mechanical inoculation protocol has been internally optimized by the inventors and used with two distinct RB-TSWV strains. The cotyledon or one to two true leaves of the young plants was gently rubbed with inoculum. The inoculation was repeated every week for three weeks.

Scorings are preformed 15 days and 21 days after the first inoculation. The main symptoms scored are Ringspot and Mosaic/Mottle following by curling, stunting, chlorosis and lesions.

Consistent results have been found for these RB-TSWV strains on the wild accession Line 111.

Inoculation by thrips (Frankliniella occidentalis) has also been tested and compared with mechanical inoculation.

On susceptible controls, both inoculation methods (thrips-mediated and mechanical) show 100% of infected plants, thus validating the thrips-mediated inoculation, i.e. it demonstrated that thrips successfully acquired and transmitted the virus with the protocols used.

The inventors have also concluded that mechanical inoculation appears to be stronger than thrips-mediated inoculation, such that resistance identified through mechanical inoculation is expected to translate into resistance to natural infection by thrips.

### Elisa

ELISA test has been performed in all inoculated plants to evaluate the presence of the virus in the plants, generally 4 weeks after inoculation.

### Example 2: Sequencing the Viral Strains

The NSm gene has been sequenced for the different RB-TSWV strains. Two strains, representing the three most common mutations have been retained (see Table 1) including the well-known C118 (Table 1).

TABLE 1
RB-TSWV strains used in this test.

| | | NSm amino acid position mutation | | |
|---|---|---|---|---|
| | | C118F | C118Y | V137I |
| Type of | RB-TSWV strain 1 | X | | X |
| strains | RB-TSWV strain 2 | | X | |

### Example 3: Identification of a Resistant Line and Comparison with Other Resistant Plants

The inventors have screened more than 50 different wild accessions of the Solanum gender, with a view to identifying a potential source of resistance to TSWV, including the RB-TSWV.

These plants were mechanically inoculated as described in example 1, with viral TSW strains, including the two RB-TSWV strains disclosed in example 1.

Plants showing less symptoms were retained and re-tested, including with an inoculation protocol with thrips, which is more similar to natural conditions.

An accession, namely line 111, showed an important and reproducible resistance in these initial tests and was retained for further characterization.

To confirm the resistance of the identified line, Elisa tests have been performed in inoculated plants with a wild type TSW strain, and the two RB-TSWV isolates described in example 2. Tests have been performed on plants of the identified Line 111, as well as on plants comprising the sw-5b gene (wild type) and plants not comprising said gene at all.

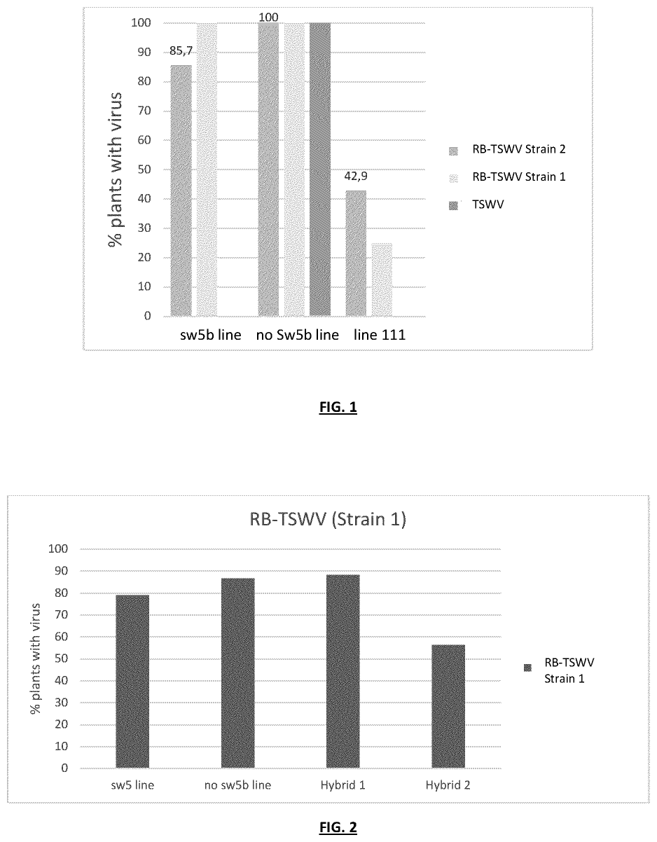

The results are illustrated in FIG. 1.

As can be inferred from this figure, for the RB-TSWV strains RB-TSWV strain 1 and strain 2, between 60% to 75% reduction in infected plants has been observed in the accession 111 compared to the 2 control lines (sw5b line and No-sw5b line) that show 100% infection.

With a wild type TSWV strain, not resistance breaking, both line 111 and the plants comprising the sw-5b gene show complete resistance (no infected plants).

These results confirm that the wild type sw-5b gene provides resistance against wildtype TSWV strains only but not against RB-TSWV strains. The identified line 111 is resistant against all types of TSWV, either wildtype strains or Resistance breaking strains, confirming the wide applicability of the resistance present in this strain.

The resistance of two hybrids (Centus and Performer) from ISI Sementi, comprising an allele of sw-5b gene allegedly conferring resistance to RB-TSWV, was tested with the RB-TSWV strain 1 disclosed in Example 2. The plants were inoculated three times at seedling stage with RB-TSWV strain 1. Phenotypic scoring at 3 weeks post inoculation and 4 weeks post inoculation was carried out, as well as TSWV ELISA in order to determine the percentage of plants with or without the disease.

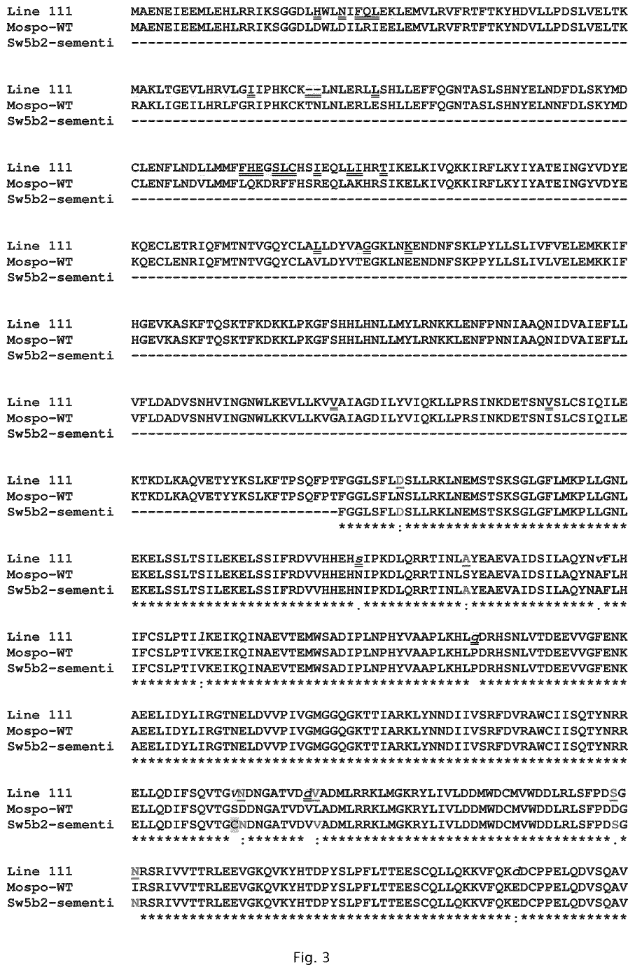

The results are illustrated in FIG. 2 and show that these hybrids do not exhibit a high level of resistance.

### Example 4: Sequencing the Resistance Protein

Sw5b gene from the wild accession Line 111 disclosed in example 3, has been sequenced and compared with the sw5b gene from the Mospomor line and the sequence sw5b2 disclosed by Isi Sementi in application WO2015/090468.

The Mospomor line is homozygous for sw5b and causes resistance to TSWV strains. The Sementi patent published a partial sequence of sw5b gene called Sw5b2. This patent claimed a new sequence of sw5b that allegedly brings intermediate resistance to an Italian resistance breaking strain.

These hybrids have been tested with the RB-TSWV strain 1 disclosed in Example 2 and do not show a high level of resistance (see FIG. 2).

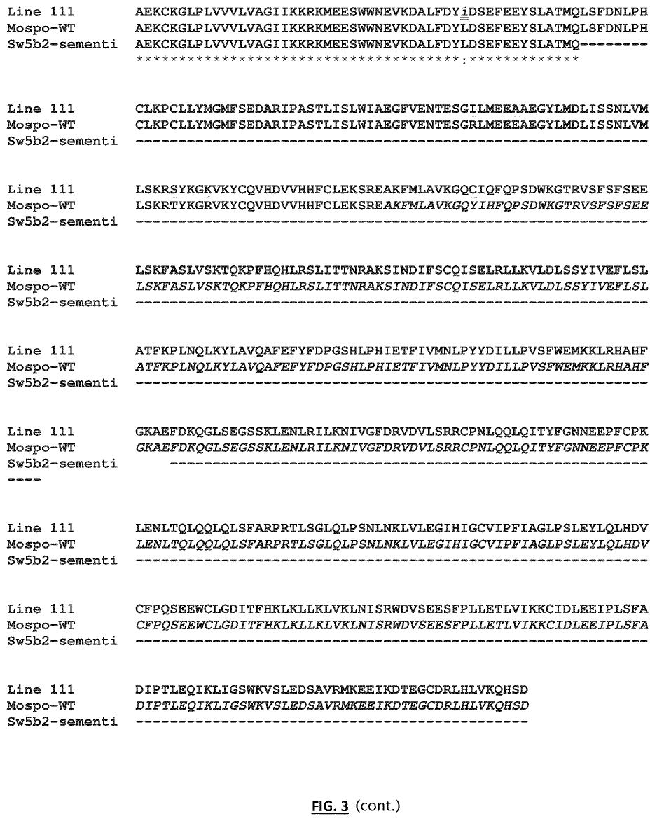

Interestingly, the sw5b gene from Line 111 presents multiple mutations compared to the WT sw5b gene and patented sequence (See FIG. 3 for a sequence alignment on this region). Six mutations are conserved with the Sw5b2 sequence disclosed by Sementi (underlined) and eight are new mutations specific to sw5b from Line 111 (italics, lower case in FIG. 3) in this region.

### Example 5: Confirmation of Recognition Between NSm Protein of RB-Strains and SW5B Protein Variant

The full sw5b sequence of Line 111 has been used to test the recognition between this new version of sw5b and frequent mutations observed in the RB-TSWV Nsm protein, especially the two RB-TSWV strains disclosed in example 2.

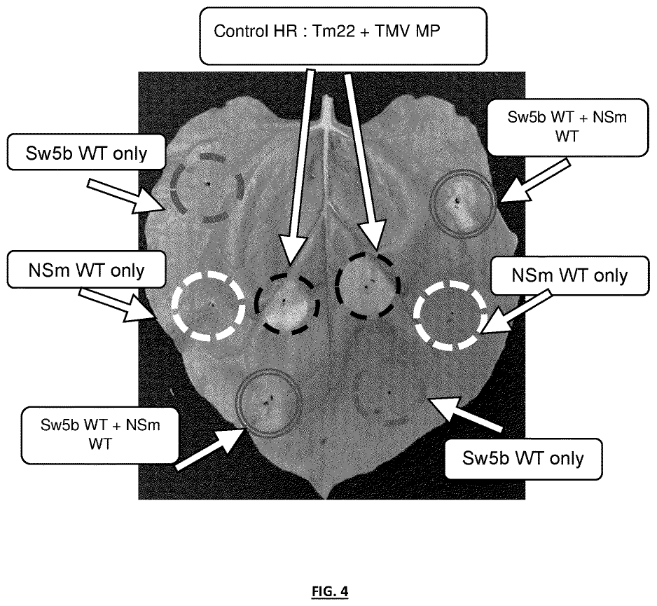

Transient assays with co-agroinfiltration have been performed on Nicotiana benthamiana leaves. The suitably of the test was first tested with the wild type Sw-5b protein and NSm protein, as disclosed in Hallwass et al. The movement protein of TMV (MP-TMV) and the TM2-2 protein, known to trigger HR were used as positive control. The results are illustrated in FIG. 4.

The detection of HR entirely matches the presence of the expected between Sw-5b protein and NSm protein.

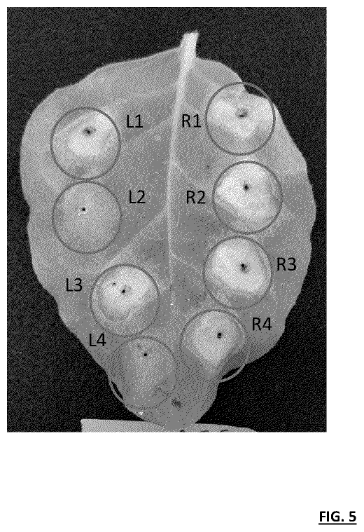

The same protocol was then used for testing interaction between the NSm protein of 4 different TSWV strains, namely the wild type sequence and a known variant not identified as resistance-breaking, and the two RB-TSWV characterised in example 2, and the wild type Sw-5b protein or the variant allele identified in Line 111. The results are illustrated in FIG. 5.

These transient assay results showed HR for all NSm variants and NSm WT (spot 1) with the Sw5b sequence from the wild accession 111 (R). In presence of sw5b from the line Mospomor, binding recognition (or HR) is lost in the presence of the NSm variant with a mutation in C118Y (spot L2) and the NSm variant with mutations in V137I and V153I (spot L4), known as resistance-breaking strains.

These results confirm that the resistance observed in accession 111 (example 3) comes indeed from the allele of the sw-5b gene of this accession.

### Example 6: Transfer of the Gene in Commercial Background

The inventors succeeded in crossing resistant plants of the line 111, as identified in the preceding example, with S. Lycopersicum plants.

The following steps have been carried out:
1. F1 plants were produced by crossing a plant from the resistant line 111 with an elite line from processing tomato. Embryo rescue was performed as the seeds were not viable.
2. F1 plants were confirmed to be hybrid between the S. lycopersicum genome and the wild accession not lycopersicum, using a molecular marker.
3. Crosses were made with F1 from line 111 to obtain BC1F1 plants. Embryo rescue was performed to obtain the BC1F1 plants, as the plants were still not viable at this stage.

Although there is a protocol for tomato Embryo rescue for breeding scheme using S. lycopersicum genome and the wild accessions, different adaptations were necessary to obtain hybrid plants.

BC1F1 plants were tested for resistance.

The test has been done with a RB-TSWV strain 1, similar to those circulating inter alia in South America, in Growth chamber. 16 plants (coming from one BC1 embryo-rescued plant) per genotypes have been sowed.

Inoculations on tomato plants were done twice. Scorings were done 25 days and 32 days post inoculation and ELISA at 35 dpi.

Inoculations on tomato plants were done twice (27 and 38 days after sowing). Scorings were done 24 days and 31 days post inoculation (first inoc). ELISA tests were performed 31 days after inoculation. Mospomor and EP7 were used as susceptible controls.

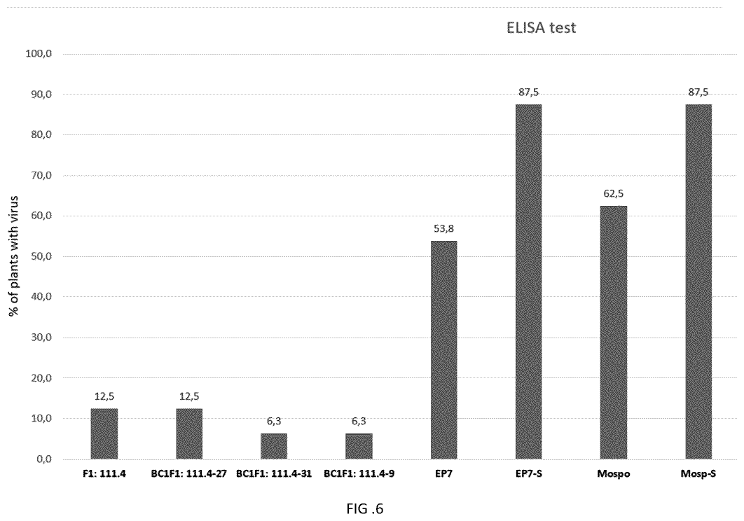

All F1 (111.4), BC1F1 and controls are coming from cutting, except controls labelled as “Mosp-S” and “EP7-S” on FIG. 6 which are from seeds. The BC1F1 tested are 111.4-9, 111.4-27 and 111.4-31. They were obtained by embryo rescue and cutting. Results are illustrated in FIG. 6, and confirm that the resistance phenotype has been duly transferred to the progeny.

The BC1F1 line 1Nov. 4, 2031 has been selected based on molecular confirmation of the cross and for it high level of resistance. This line has been used to be crossed with multiple S. lycopersicum sources. BC2F2 populations were generated and used for phenotyping and pathology assays.

Further selfings are carried out in order to obtain S. lycopersicum plants, having agronomical traits of interest and resistance to TSWV, including RB-TSWV strains.

### Example 7: RB-TSWV Resistance in Seedling Assay in S. lycopersicum Background

In order to evaluate the level of RB-TSWV resistance of this source in tomato, a grow chamber phenotyping test has been done on BC2F2 populations.

### Viral Strains:

The strain1 (South America) has been used for this example.

### Viral Inoculation:

The RB-TSWV strain was multiplied in the leaves of susceptible plants. The symptomatic leaves are harvested and stored in −80 °C.

The optimal results are obtained by using Datura plant as a maintainer. Fresh datura plants are mechanically inoculated with frozen ground tissue infected with the RB-TSWV strain. The new infected datura leaves correspond to the 3^{rd} passage inoculum. Tomato plants are inoculated with the fresh inoculated Datura leaves. This corresponds to the 3^{rd} passage (1^{st} passage: original material frozen in-80 °C, 2^{nd} passage: Datura leaves, 3^{rd} passage: Tomato plants). A second inoculation could be done.

Mechanical inoculation protocol has been internally optimized by the inventors and used. The cotyledon of the young plants was gently rubbed with inoculum. The inoculation was repeated every week for two weeks.

Scorings are preformed 15 days and 21 days after the first inoculation. The main symptoms scored are Ringspot and Mosaic/Mottle following by curling, stunting, chlorosis and lesions.

The controls in this trial are as follows:

Susceptible check:
Mospomor: homozygous sw5b inbreed
Hybrid carrying sw5b Heterozygous

Source of resistance: 111

### ELISA

ELISA test has been performed in all inoculated plants to evaluate the presence of the virus in the plants, generally 4 weeks after inoculation.

### Results

TABLE 2
reports the results of the test including scoring (foliar symptoms)
21 dpi and ELISA assay.

| | | | | ELISA | | |
|---|---|---|---|---|---|---|
| | | av. stand | | number | number | % |
| | number | deviation | standard | plants | plants | resistant |
| | of plants | positive | deviation | Positive | negative | plants |
| 111 | 28 | 5.7 | 2.8 | 18 | 9 | 32.1 |
| Mospomor | 33 | 4.3 | 2 | 33 | 0 | 0.0 |
| Hybrid | 17 | 3.6 | 2.8 | 17 | 0 | 0.0 |
| BC2F2 | 29 | 6.3 | 2.7 | 18 | 11 | 37.9 |

The results of this test show that the resistant phenotype of the source 111 has been transferred in S. lycopersicum background. In the generation BC2F2, 11 out of 29 plants are free of RB-TSWV approximately 4 weeks after inoculation.

The present invention is more specifically directed to the following aspects:
1. A Sw-5b protein recognizing the viral movement protein NSm of TSWV, wherein said protein has at least 90% sequence identity to the wild type Sw-5b protein (SEQ ID NO:1) and comprises at least one, preferably, 2, 3 or 4 variations with respect to SEQ ID NO:1 found at positions 23, 26, 28, 29, 30, 75, 82, 83, 90, 134, 135, 136, 138, 139, 140, 143, 147, 148, 151, 203, 209, 214, 325, 351, 448, 522, 622 and/or 759.
2. The Sw-5b protein according to aspect 1, wherein the at least one variation is chosen amongst the following mutations with respect to SEQ ID NO:1: D23H, D26N, L28F, R29Q, I30L, R75I, T82DEL, N83DEL, E90L, L134F, Q135H, K136E, R138S, F139L, F140C, R143I, A147L, K148I, S151T, V203L, E209G, E214K, G325V, I351V, N448S, P522Q, V622D and L759I.
3. The Sw-5b protein according to aspect 1 or 2, comprising at least one, preferably two, three or the following 4 variations with respect to SEQ ID NO:1: N448S; P522Q; V622D and L759I.
4. The Sw-5b protein according to any one of aspects 1 to 3, comprising at least the following 4 variations with respect to SEQ ID NO:1: N448S; P522Q; V622D and L759I.
5. The Sw-5b protein according to any one of aspects 1 to 4, further comprising one or more of the following variations with respect to SEQ ID NO:1: A477V; V489L; D659S and E707D.
6. The Sw-5b protein according to any one of aspects 1-5, further comprising one or more of the following mutations with respect to SEQ ID NO:1: N393D; S461A; D614N; L623V and I661N; and potentially also S613V or S613C.
7. The Sw-5b protein according to any one of aspects 1-6, comprising the following variations with respect to SEQ ID NO:1: N393D; N448S; S461A, A477V; V489L; P522Q; D659S, D614N, V622D; L623V, I661N, E707D and L759I.
8. The Sw-5b protein recognizing the viral movement protein NSm of TSW, according to aspect 1, comprising at least one of the following variations with respect to SEQ ID NO:1: K136E; R138S; F139L; F140C; R143I and A147L.
9. The Sw-5b protein according to aspect 8, comprising at least four of the following variations with respect to SEQ ID NO:1: K136E; R138S; F139L; F140C; R143I and A147L; preferably at least 5 of these variations, or the 6 variations.
10. The Sw-5b protein according to aspect 8 or 9, further comprising the variation D137G.
11. The Sw-5b protein according to any one of aspects 8 to 10, further comprising one or more of L134F; Q135H, K148I and S151T.
12. The Sw-5b protein according to any one of aspects 1 to 11, further comprising a deletion of amino acids 82 (T) and/or 83 (N) of SEQ ID NO:1.
13. The Sw-5b protein according to any one of aspects 1 to 12 further comprising the variation K319E with respect to SEQ ID NO:1.
14. The Sw-5b protein according to any one of aspects 1 to 13, wherein the amino acids corresponding to L33 and/or R927 in SEQ ID NO:1 are not modified.
15. The Sw-5b protein according to any one of aspects 1 to 14, having at least 92%, preferably at least 94% sequence identity with SEQ ID NO:1, over the whole sequence.
16. The Sw-5b protein according to any one of aspects 1-15, having at least 95% sequence identity with SEQ ID NO:1.
17. The Sw-5b protein according to any one of aspects 1-16, having the sequence set forth in SEQ ID NO:3, or a sequence having more than 99% sequence identity with SEQ ID NO:3.
18. The Sw-5b protein according to any one of aspects 1-17, recognizing and interacting with the viral movement protein NSm of resistance-breaking TSWV strains, including strains comprising the C118Y mutation and strains comprising the V137I et V153I mutations.
19. The Sw-5b protein according to any one of aspects 1-18, recognizing and interacting with the viral movement protein NSm of TSWV strains, known as RB-TSWV, which do not interact with SEQ ID NO:1.
20. The Sw-5b protein according to any one of aspects 1-19, conferring resistance to TSWV infection in tomato, peppers, potato and/or lettuce, including resistance-breaking TSWV strains comprising the C118Y mutation and strains comprising the V137I et V153I mutations.
21. A resistance gene encoding a Sw-5b protein according to any one of aspects 1-20, conferring resistance to TSWV infection in tomato plants.
22. A nucleic acid construct comprising a sequence encoding a Sw-5b protein according to any one of aspects 1-20 or comprising a resistance gene according to claim 21, preferably under the control of a promoter, e.g. a vector, a plasmid or a T-DNA plasmid.
23. A nucleic acid construct according to aspect 21 or 22, comprising a sequence having at least 75% sequence identity to SEQ ID NO:11, preferably at least 90%, more preferably at least 99%.
24. A Solanum lycopersicum, N. tabacum, N. benthamiana, or Capsicum annuum cell, expressing transiently or constitutively a Sw-5b protein according to any one of aspects 1-20.
25. A Solanum lycopersicum, N. tabacum, N. benthamiana, or Capsicum annuum cell, comprising a resistance gene according to claim 21 or a nucleic acid construct according to aspect 22 or 23, preferably within its genomic DNA, homozygously or heterozygously.
26. The cell according to aspect 25 or a tissue culture of said cells, wherein the cells are derived from embryos, protoplasts, meristematic cells, callus, pollen, leaves, anthers, stems, petioles, roots, root tips, seeds, flowers, cotyledons, and/or hypocotyls, and contain in their genome said nucleic acid construct or said resistance gene.
27. A Solanum lycopersicum, N. tabacum, N. benthamiana, or Capsicum annuum plant, resistant against TSWV infection, including by resistance-breaking strains, wherein said plant comprises in its genome, preferably on chromosome 9, a resistance gene according to aspect 21, homozygously or heterozygously.
28. A S. lycopersicum plant resistant to TSWV, comprising an allele of the Sw-5b gene encoding a Sw-5b protein having at least 90% sequence identity to the wild type Sw-5b protein (SEQ ID NO:1) and comprising at least one of the following variations with respect to SEQ ID NO:1: substitution D23H, substitution D26N, substitution L28F, substitution R29Q, substitution I30L, substitution R75I, deletion T82DEL, deletion N83DEL, substitution E90L, substitution L134F, substitution Q135H, substitution K136E, substitution R138S, substitution F139L, substitution F140C, substitution R143I, substitution A147L, substitution K148I, substitution S151T, substitution V203L, substitution E209G, substitution E214K, substitution G325V, substitution I351V, substitution N448S, substitution P522Q, substitution V622D and substitution L759I.
29. The S. lycopersicum plant resistant to TSWV according to aspect 28, comprising an allele of the Sw-5b gene encoding a Sw-5b protein comprising at least one of the following variations with respect to SEQ ID NO:1: K136E; R138S; F139L; F140C; R143I and A147L, preferably at least 2, 3 or 4 of these variations.
30. The S. lycopersicum plant resistant to TSWV according to aspect 29, comprising at least four of the variations K136E; R138S; F139L; F140C; R143I and A147L, preferably at least 5 of these variations, or the 6 variations
31. The S. lycopersicum plant resistant to TSW according to aspect 28, comprising an allele of the Sw-5b gene encoding a Sw-5b protein comprising at least one of the following variations with respect to SEQ ID NO:1: N448S; P522Q; V622D and L759I, and preferably two or three.
32. The S. lycopersicum plant according to aspect 31, comprising the following four variations with respect to SEQ ID NO:1: N448S; P522Q; V622D; and L759I.
33. The S. lycopersicum plant according to any one of aspects 28 to 32, wherein said allele of the Sw-5b gene is obtained by gene editing, base-editing or prime-editing techniques, preferably by mutagenesis, by TILLING and/or CRISPR/Cas system.
34. A plant part of a S. lycopersicum plant according to any one of aspects 27-33, in particular seeds, explants, reproductive material, scion, cutting, seed, fruit, root, rootstock, pollen, ovule, embryo, protoplast, leaf, anther, stem, petiole or flowers, wherein said plant part comprises at least one cell according to aspect 24, 25 or 26.
35. A S. lycopersicum seed, which can be grown into a S. lycopersicum plant according to any one of claims 27-33 or which comprises at least one cell according to any one of aspects 24-26.
36. A method for obtaining transgenic S. lycopersicum plants resistant to TSWV, including RB-TSWV strains, comprising:
   Obtaining a construct comprising a sequence encoding a Sw-5b protein according any one of aspects 1-20 or comprising a resistance gene according to claim 21,
   Introducing said construct into a S. lycopersicum cell,
   Regenerating a transgenic plant;
   Optionally propagating the obtained plant.
37. A method of producing a S. lycopersicum plant resistant to TSWV, including RB-TSWV strains, comprising the introduction of at least one mutation in the Sw-5b gene on chromosome 9 of a Sw-5b-comprising S. lycopersicum plant, wherein said at least one mutation are introduced by mutagenesis, by TILLING, by genome editing, base-editing or prime editing, in particular by a technique selected from N-methyl-N-nitrosourea (MNU) mutagenesis, sodium azide (NaN3, SA) mutagenesis, oligonucleotide directed mutagenesis (ODM), Zinc finger nuclease (ZFN) technology, Transcription Activator-Like Effector Nucleases (TALENs) the CRISPR/Cas system, Cas9, Cas21a, engineered meganuclease, re-engineered homing endonucleases and DNA guided genome editing; and
wherein said mutation gives rise to at least the following amino acid substitution or deletion in the protein encoded by the Sw-5b gene: substitution D23H, substitution D26N, substitution L28F, substitution R29Q, substitution I30L, substitution R75I, deletion T82DEL, deletion N83DEL, substitution E90L, substitution L134F, substitution Q135H, substitution K136E, substitution R138S, substitution F139L, substitution F140C, substitution R143I, substitution A147L, substitution K148I, substitution S151T, substitution V203L, substitution E209G, substitution E214K, substitution G325V, substitution I351V, substitution N448S, substitution P522Q, substitution V622D and substitution L759I.
38. The method of aspect 37, wherein said mutation gives rise to:
   at least one of the following amino acid substitutions in the protein encoded by the Sw-5b gene: N448S; P522Q; V622D; and L759I; and preferably 2, 3 or the 4 substitutions, or
   at least one of the following amino acid substitutions in the protein encoded by the Sw-5b gene: K136E; R138S; F139L; F140C; R143I; A147L, and preferably at least 2, 3, or at least 4 of the substitutions.
39. A method for breeding a S. lycopersicum plant resistant to TSWV, including RB-TSWV strains, comprising:
a. Crossing a S. lycopersicum plant comprising a resistance gene according to aspect 21 with an initial S. lycopersicum plant devoid of said resistance gene,
b. Selecting in the progeny thus obtained, a plant bearing the resistance gene,
C. Optionally self-pollinating one or several times the plant obtained at step (b) and selecting in the progeny thus obtained a plant bearing the resistance gene.
40. A method of producing a S. lycopersicum plant resistant to TSWV, comprising:
a. obtaining a part of a plant according to any one of aspects 27-33,
b. vegetatively propagating said plant part to generate a plant from said plant part.
41. Use of a sequence according to aspect 21 or a construct according to aspect 22 or 23, for conferring resistance against TSWV, including to resistance-breaking TSWV strains, to a S. lycopersicum plant or for obtaining transgenic S. lycopersicum plants resistant to TSWV.
42. Use of a plant or seed of S. lycopersicum, or a part thereof or a progeny thereof, bearing the resistance gene according to aspect 21 or a construct according to aspect 22 or 23, as a breeding partner in a breeding program for conferring resistance to TSWV to S. lycopersicum plants, including to resistance-breaking TSWV strains.
43. A method for improving the yield of tomato plants or for reducing the loss on tomato production in an environment infested or likely to be infested by TSWV, especially by resistance-breaking strains of TSWV, comprising growing tomato plants comprising in their genome the resistance gene according to aspect 21.
44. A method for reducing the loss on tomato production in conditions of TSWV infestation, especially by resistance-breaking strains of TSWV, comprising growing a tomato plant comprising in its genome the resistance gene according to aspect 21.
45. A method of producing tomatoes comprising:
   a. growing a S. lycopersicum plant according to any one of aspects 27-33;
   b. allowing said plant to set fruit; and
   c. harvesting fruit of said plant, preferably at pre-mature or mature stage.
46. A method for identifying, detecting and/or selecting S. lycopersicum plants resistant to resistance-breaking strains of TSWV, amongst plants resistant to TSWV, said method comprising the detection of an allele of the Sw-5b gene in the genome of said plants, wherein said allele comprises at least one variation giving rise to at least one amino acid substitution in the Sw-5b protein encoded by the Sw-5b gene with respect to the wild type sequence SEQ ID NO:1, chosen in the group constituted by the D23H, D26N, L28F, R29Q, I30L, R75I, T82DEL, N83DEL, E90L, L134F, Q135H, K136E, R138S, F139L, F140C, R143I, A147L, K148I, S151T, V203L, E209G, E214K, G325V, I351V, N448S, P522Q, V622D and L759I substitutions or deletions in the Sw-5b protein,
and preferably in the group constituted by K136E; R138S; F139L; F140C; R143I; A147L, N393D; N448S; S461A, A477V; V489L P522Q; D659S, D614N, V622D; L623V, I661N, E707D, and L759I substitutions in the Sw-5b protein.
47. A molecular marker for identifying, detecting and/or selecting S. lycopersicum plants resistant to resistance-breaking strains of TSWV, amongst plants resistant to TSWV, wherein said marker is less than 50 nucleotide long, is at least 95% identical to the corresponding portion of SEQ ID NO:10, and is able to discriminate at least one of the following variations in the sequence encoded by SEQ ID NO:11 with respect to SEQ ID NO:10: D23H, D26N, L28F, R29Q, I30L, R75I, T82DEL, N83DEL, E90L, L134F, Q135H, K136E, R138S, F139L, F140C, R143I, A147L, K148I, S151T, V203L, E209G, E214K, G325V, I351V, N448S, P522Q, V622D and L759I; and preferably K136E; R138S; F139L; F140C; R143I; A147L, N393D; N448S; S461A, A477V; V489L, P522Q; D659S, D614N, V622D; L623V, I661N, E707D, and L759I.
48. The molecular marker according to aspect 47, which can discriminate between a sequence encoding a Sw-5b protein having at least one of the following variations with respect to SEQ ID NO:1: K136E; R138S; F139L; F140C; R143I and A147L.
49. The molecular marker according to aspect 47, which can discriminate between a sequence encoding a Sw-5b protein having at least one of the following variations with respect to SEQ ID NO:1: N448S, P522Q, V622D and L759I.
50. The molecular marker according to aspect 47, 48 or 49, wherein said marker is an amplification probe or a detection probe.

### REFERENCES

Crescenzi, A., Fanigliulo, A., and Viggiano, A. (2015). RESISTANCE BREAKING TOMATO SPOTTED WILT VIRUS ISOLATES ON RESISTANT TOMATO CULTIVARS IN ITALY. Acta Hortic. 1069, 95-98
Hallwass M., de Oliveira A. S., de Campos Dianese E., Lohuis D., Boiteux L. S., Inoue-Nagata A. K., et al. (2014). The Tomato spotted wilt virus cell-to-cell movement protein (NSm) triggers a hypersensitive response in Sw-5-containing resistant tomato lines and in Nicotiana benthamiana transformed with the functional Sw-5b resistance gene copy. Mol. Plant Pathol. 15 871-880.
Huang, H., Huang, S., Li, J., Wang, H., Zhao, Y., Feng, M., Dai, J., Wang, T., Zhu, M. and Tao, X. (2021) Stepwise artificial evolution of an Sw-5b immune receptor extends its resistance spectrum against resistance-breaking isolates of Tomato spotted wilt virus. Plant Biotechnol. J., 1-13
Li et al., 2019. A Plant Immune Receptor Adopts a Two-Step Recognition Mechanism to Enhance Viral Effector Perception. Mol. Plant. 12, 248-262
Peiro A., Canizares M. C., Rubio L., Lopez C., Moriones E., Aramburu J., et al. (2014). The movement protein (NSm) of Tomato spotted wilt virus is the avirulence determinant in the tomato Sw-5 gene-based resistance. Mol. Plant Pathol. 15 802-813.
Seong et al., 2022. Evolution of NLR resistance genes with noncanonical N-terminal domains in wild tomato species. New Phytologist (2020) 227:1530-1543
Stevens J. M. 1964. Tomato Breeding. Project report W-Vv1, Department of Agricultural Technical Services, Republic of South Africa
Wang, J., Chen, T., Han, M., Qian, L., Li, J., Wu, M., Han, T., Cao, J., Nagalakshmi, U., Rathjen, J. P., Hong, Y., and Liu, Y. 2020. Plant NLR immune receptor Tm-22 activation requires NB-ARC domain-mediated self-association of CC domain. PLOS Pathog 16: e1008475.

1. A S. lycopersicum plant resistant to TSWV, comprising an allele of the Sw-5b gene encoding a Sw-5b protein having at least 90% sequence identity to the wild type Sw-5b protein (SEQ ID NO:1) and comprising at least five of the following variations with respect to SEQ ID NO:1: substitution D23H, substitution D26N, substitution L28F, substitution R29Q, substitution I30L, substitution R75I, deletion T82DEL, deletion N83DEL, substitution E90L, substitution L134F, substitution Q135H, substitution K136E, substitution R138S, substitution F139L, substitution F140C, substitution R143I, substitution A147L, substitution K148I, substitution S151T, substitution V203L, substitution E209G, substitution E214K, substitution G325V, substitution I351V, substitution N448S, substitution P522Q, substitution V622D and substitution L759I.

2. The S. lycopersicum plant resistant to TSWV according to claim 1, comprising an allele of the Sw-5b gene encoding a Sw-5b protein comprising at least three of the following variations with respect to SEQ ID NO:1: K136E; R138S; F139L; F140C; R143I and A147L.

3. The S. lycopersicum plant according to claim 1, comprising the following four variations with respect to SEQ ID NO:1: N448S; P522Q; V622D; and L759I.

4. The S. lycopersicum plant according to claim 1, comprising an allele of the Sw-5b gene encoding a Sw-5b protein wherein the amino acids corresponding to L33 and/or R927 in SEQ ID NO:1 are not modified.

5. The S. lycopersicum plant according to claim 1, wherein said allele of the Sw-5b gene encodes a Sw-5b protein having at least 95% sequence identity to the wild type Sw-5b protein (SEQ ID NO:1).

6. (canceled)

7. The S. lycopersicum plant according to claim 1, wherein said plant is resistant to TSWV, including resistance-breaking TSWV strains comprising the C118Y mutation and strains comprising the V137I et V153I mutations.

8. A plant part of a S. lycopersicum plant according to claim 1, wherein said plant part comprises at least one cell comprising said allele of the Sw-5b gene.

9. A S. lycopersicum seed, which can be grown into the S. lycopersicum plant according to claim 1.

10. A Sw-5b protein recognizing the viral movement protein NSm of TSWV, wherein said protein has at least 90% sequence identity to the wild type Sw-5b protein (SEQ ID NO:1) and comprises at least one, with respect to SEQ ID NO:1 found at positions 23, 26, 28, 29, 30, 75, 82, 83, 90, 134, 135, 136, 138, 139, 140, 143, 147, 148, 151, 203, 209, 214, 325, 351, 448, 522, 622 and/or 759.

11. The Sw-5b protein according to claim 10, wherein the at least one variation is chosen amongst the following mutations with respect to SEQ ID NO:1: D23H, D26N, L28F, R29Q, I30L, R75I, T82DEL, N83DEL, E90L, L134F, Q135H, K136E, R138S, F139L, F140C, R143I, A147L, K148I, S151T, V203L, E209G, E214K, G325V, I351V, N448S, P522Q, V622D and L759I.

12. The Sw-5b protein according to claim 10, comprising at least one, of the following 4 variations with respect to SEQ ID NO:1: N448S; P522Q; V622D and L759I.

13. The Sw-5b protein according to claim 10, comprising the following variations with respect to SEQ ID NO:1: N393D; N448S; S461A, A477V; V489L; P522Q; D659S, D614N, V622D; L623V, 1661N, E707D and L759I.

14. The Sw-5b protein according to claim 10, comprising at least three of the following variations with respect to SEQ ID NO:1: K136E; R138S; F139L; F140C; R143I and A147L.

15. (canceled)

16. The Sw-5b protein according to claim 10, having at least 92%, sequence identity with SEQ ID NO:1, over the whole sequence; or having the sequence set forth in SEQ ID NO:3, or a sequence having more than 99% sequence identity with SEQ ID NO:3.

17. (canceled)

18. The Sw-5b protein according to claim 10, conferring resistance to TSWV infection in tomato, peppers, potato and/or lettuce, including resistance-breaking TSWV strains comprising the C118Y mutation and strains comprising the V137I et V153I mutations.

19. A resistance gene encoding the Sw-5b protein according to claim 10, conferring resistance to TSWV infection in tomato plants.

20.-21. (canceled)

22. A Solanum lycopersicum, Nicotinia tabacum, Nicotinia benthamiana, or Capsicum annuum cell, comprising the resistance gene according to claim 19, homozygously or heterozygously.

23. A Solanum lycopersicum, N. tabacum, N. benthamiana, or Capsicum annuum plant, resistant against TSWV infection, including by resistance-breaking strains,
wherein said plant comprises in its genome, the resistance gene according to claim 19, homozygously or heterozygously.

24. A method for obtaining transgenic S. lycopersicum plants resistant to TSWV, including RB-TSWV strains, comprising:
Obtaining a construct comprising the sequence encoding a Sw-5b protein according to claim 10 or comprising the resistance gene encoding the Sw-5b protein according to claim 10, conferring resistance to TSWV infection in tomato plants,
Introducing said construct into a S. lycopersicum cell,
Regenerating a transgenic plant;
Optionally propagating the obtained plant.

25.-26. (canceled)

27. A method for breeding a S. lycopersicum plant resistant to TSWV, including RB-TSWV strains, comprising:
a. Crossing a S. lycopersicum plant comprising the resistance gene according to claim 19 with an initial S. lycopersicum plant devoid of said resistance gene,
b. Selecting in the progeny thus obtained, a plant bearing the resistance gene,
c. Optionally self-pollinating one or several times the plant obtained at step (b) and selecting in the progeny thus obtained a plant bearing the resistance gene.

28.-37. (canceled)
